(19) United States
(12) Patent Application Publication
(10) Pub. No.: US 2025/0384987 A1
(43) Pub. Date: Dec. 18, 2025

(54) MEDICAL PRESCRIPTION REVIEW AND DECISION SUPPORT SYSTEM

(71) Applicant: Alto Pharmacy LLC, San Francisco, CA (US)

(72) Inventors: Kelsey Kacius, Larkspur, CA (US); Nilanjana Adlakha, San Jose, CA (US); Sijia Shen, San Francisco, CA (US); Vicky Cao, Lake Oswego, OR (US); Sudarshan Seshadri, San Francisco, CA (US); Nir Sheep, Kirkland, WA (US); Daniel Katz, San Mateo, CA (US); Sierra Brown, San Francisco, CA (US)

(21) Appl. No.: 19/239,655

(22) Filed: Jun. 16, 2025

Related U.S. Application Data
(60) Provisional application No. 63/660,396, filed on Jun. 14, 2024.

(51) Int. Cl. G16H 20/10; G16H 10/60; G16H 70/40

(52) U.S. Cl. CPC G16H 20/10; G16H 10/60; G16H 70/40

(57) ABSTRACT

A Decision Support System for prescription verification may include software and/or hardware configured to supplement analysis and decision-making in the workflow of a pharmacist and/or take action regarding certain steps in the prescription fulfillment process. The Decision Support System may include a plurality of software modules each configured to provide information to a user for a particular review, evaluation, or check regarding the Prescription Verification process. One or more the software modules of the Decision Support System may use or be implemented as an artificial intelligence (AI) module or algorithm, e.g., one or more decision trees, predictive models, large language models (LLMs), RAG enhanced LLMs, or rules-based engines. Decision Support System is configured to check information regarding the patient receiving the medication, the provider prescribing the medication, and the medication itself to ensure data accuracy and to determine whether it is safe to dispense the medication to the patient.

### CROSS-REFERENCES

The following applications and materials are incorporated herein by reference, in their entireties, for all purposes: U.S. Provisional Patent Application Ser. No. 63/660,396, filed Jun. 14, 2024.

### FIELD

This disclosure relates to systems and methods for assisting pharmacies in the key task of reviewing medical prescriptions and obtaining clarifications from the prescribing physician when warranted. More specifically, the disclosed embodiments relate to artificial intelligence-enhanced review and decision support systems relating to medical prescription review and fulfillment.

### INTRODUCTION

In general, pharmacists typically have a lengthy standard operating procedure with disjointed tools to conduct their primary duty of completing a clinical review of incoming prescriptions. Pharmacists spend a considerable amount of time outside of their core competency performing tasks such as retrieving a patient address, updating erroneous prescriber data, reviewing patient dispensing history, and accessing control dispensing information via the relevant states' Prescription Drug Monitoring Program. Pharmacists must ensure no transcription errors occur between the source script and system data for patient, provider, and prescription information. Pharmacists often perform data entry to translate freeform allergy information provided by the patient. When evaluating the script itself, pharmacists often apply personal preferences beyond clinical requirements to ensure the SIG meets standards. Pharmacists also are responsible for performing mental math to confirm the number of refills remaining and validate all quantity values.

Throughout this broad array of tasks, pharmacists are frequently required to context switch between therapies and states; needing to not only keep clinical judgement top of mind for a variety of therapies, but also switch between different state-level regulatory requirements.

Even in situations where greater system integration and/or employee support reduces the need for pharmacists to do non-value-added work, the pharmacists are still responsible for reviewing the entire prescription and catching all errors. pharmacists often must reference third party sources, such as DailyMed and Clinical Pharmacology, to ensure accuracy and completeness of their review.

The pharmacy industry in general currently has no way to measure dispensing errors at scale, and there is a high amount of variability in the decision-making of individual pharmacists.

Prescription Verifications are a step required by the Board of Pharmacy for all first fills, all fills of controlled medications, and refills in situations where a patient profile component has changed since the last review. This poses a scalability problem for all pharmacies. Prescription Verifications are integral for ensuring patient safety as well as the accuracy and integrity of prescription processing. Dispensing decisions are left to the discretion and variability of human pharmacists. There is no standardized process or measurement framework to evaluate the decisions. Additionally, Prescription Verifications are the most expensive step in prescription processing.

Systems and methods are needed to improve efficiency and reduce waste in prescription fulfillment while maintaining or improving dispensing accuracy.

### SUMMARY

The present disclosure provides systems, apparatuses, and methods relating to decision support systems for medical prescription review and fulfillment.

In some examples, a prescription verification system, includes: one or more data processing systems including a memory; one or more processors; and a decision support system including one or more software programs including a plurality of instructions stored in the memory and executable by the one or more processors to: receive a request to perform a prescription verification precheck for a medical prescription, wherein the request includes prescription data associated with the medical prescription, patient data associated with a patient of the medical prescription, and provider data associated with a medical provider of the medical prescription; and perform the prescription verification precheck to determine whether the medical prescription is in a satisfactory condition to be filled or whether the medical prescription is in an unsatisfactory condition and requires review by a user prior to being filled, wherein performing the prescription verification precheck includes: verifying accuracy of the patient data, the prescription data, and the provider data; verifying the medical prescription satisfies one or more policies and regulations; and performing a prescription safety check to determine whether the medical prescription is safe to dispense to the patient based on the prescription data and the patient data.

In some examples, a computer-implemented prescription fulfillment method comprises: utilizing one or more processors of a data processing system to: receive, from a medical provider, a request to fill a medical prescription for a patient, wherein the request includes prescription data associated with the medical prescription, patient data associated with the patient, and provider data associated with the medical provider; and perform a prescription verification precheck using a decision support system including one or more software programs including a plurality of instructions stored in a memory of the data processing system and executable by the one or more processors to: verify accuracy of the patient data, the prescription data, and the provider data; verify the medical prescription satisfies one or more policies and regulations; perform a prescription safety check to determine whether the medical prescription is safe to dispense to the patient based on the prescription data and the patient data; and determine whether the medical prescription is in a satisfactory condition to be filled or whether the medical prescription is in an unsatisfactory condition and requires review by a user prior to being filled.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a flow chart depicting steps of an illustrative method for fulfilling medical prescriptions according to the present teachings.

FIG. 2 is a schematic block diagram depicting components of a decision support system in accordance with aspects of the present disclosure.

FIG. 3 is a schematic block diagram depicting components of the decision support system of FIG. 2.

FIG. 4 is a flow chart depicting steps of a prescription data validation algorithm in accordance with the present teachings.

FIG. 5 is a flow chart depicting steps of a clinical prescription safety algorithm in accordance with the present teachings.

FIG. 6 is a flow chart depicting steps of a regulatory validation algorithm in accordance with the present teachings.

FIG. 7 is a flow chart depicting steps of a patient validation algorithm in accordance with the present teachings.

FIG. 8 is a flow chart depicting steps of a provider validation algorithm in accordance with the present teachings.

FIG. 9 is a flow chart depicting steps of a pharmacy policies and standards algorithm in accordance with the present teachings.

FIG. 10 is a flow chart depicting steps of a self-assessment algorithm in accordance with the present teachings.

FIG. 11 is a block diagram depicting aspects of an illustrative machine learning model.

FIG. 12 is a schematic diagram of a data processing system suitable for use in implementing and using decision support systems of the present disclosure.

FIG. 13 is a schematic diagram of a data network system suitable for use in implementing and using decision support systems of the present disclosure.

### DETAILED DESCRIPTION

Various aspects and examples of a Decision Support System for medical prescription review and fulfillment, as well as related methods, are described below and illustrated in the associated drawings. Unless otherwise specified, a Decision Support System for medical prescription review and fulfillment in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Prescription Verification” refers to a process of reviewing all of the information available (medication type, drug interactions, allergies, dispensing history, etc.) in order to determine if the medication in question should be dispensed to the patient in question. Information is checked regarding the patient receiving the medication, the provider prescribing the medication, and the medication itself. As discussed herein, the Prescription Verification may include a “Prescription Verification Precheck” which is an entirely automated process performed by a Decision Support System to review a medical prescription for data accuracy and safety prior to review by a pharmacist. The Prescription Verification may further include a Pharmacist Verification 1 and Pharmacist Verification 2, which involves a pharmacist reviewing the prescription for data accuracy and reviewing the physical prescription to ensure the physical prescription has been filled correctly.

“Clarification” refers to the situation where clinical information is required from the provider or patient before a prescription can be filled. Clarifications fall into, but are not limited to, five categories: missing or incomplete prescription information, conflicting or unclear prescription information, potential patient safety issues, regulatory conflicts, and clinical administrative tasks. In some instances, such as potential drug allergy interactions, the patient can resolve the clarification in place of a provider. In some examples, a pharmacist may mark what they need clarified and forward that to another person (e.g., to another pharmacist) who is specifically working on getting the information (e.g., by reaching out to the prescribing doctor/clinic).

“DUR (Drug Utilization Review) Check”—A DUR Check is meant to look at how the drug is being used in order to validate safe prescribing and dispensing patterns. It focuses on bringing attention to all fields related to how the drug may interact with the patient's allergies, medical conditions, and other medications. This amounts to a safety check for dispensing this medication to the patient. A DUR Check can be triggered by a patient updating their medical information or when their insurance indicates in the billing claim response that a check is required. Whenever a pharmacist or other entity performs a Prescription Verification, they are also performing a DUR Check.

“SIG” is the portion of a prescription that provides instructions to the patient regarding how to take the prescribed medication, typically printed on the label. The SIG may include information such as the dose (e.g., 1 tablet, 2 capsules), frequency of administration (e.g., once daily, every 6 hours), route of administration (e.g., orally, topically), and duration of treatment (e.g., for 7 days).

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, a Decision Support System in accordance with the present teachings may include software and/or hardware configured to supplement analysis and decision-making in the workflow of a pharmacist and/or take action regarding certain steps in the prescription fulfillment process.

The Decision Support System may include a plurality of software modules each configured to provide information to a user (e.g., the pharmacist) for a particular review, evaluation, or check regarding the Prescription Verification process. For example, the Decision Support System may be configured to perform a Prescription Verification Precheck process prior to review of the prescription by the pharmacist. The Decision Support System may generate one or more outputs including proposed recommendations to the pharmacist or identifying potential issues which may require Clarification.

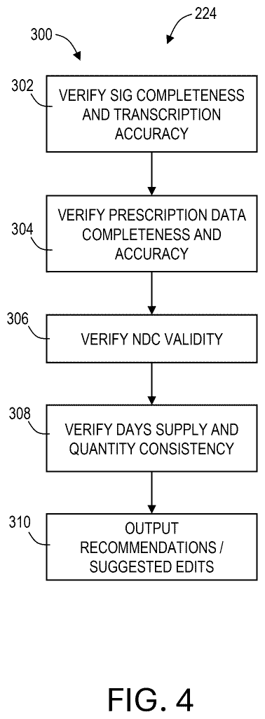

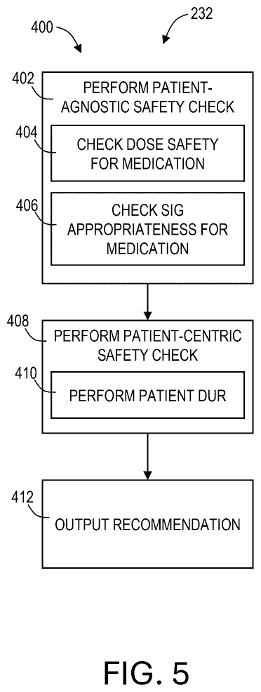

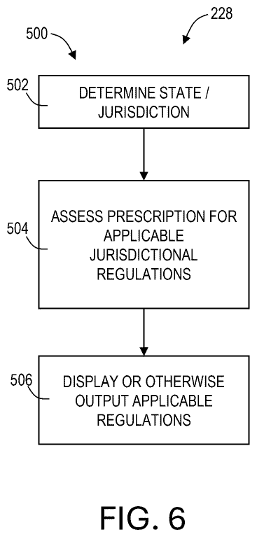

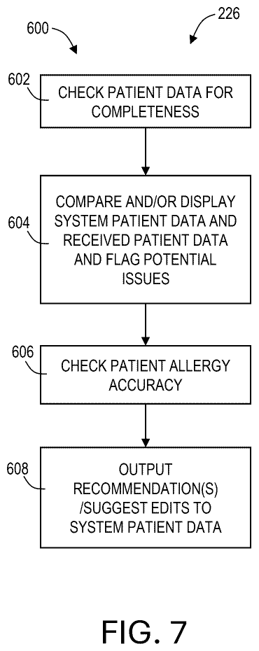

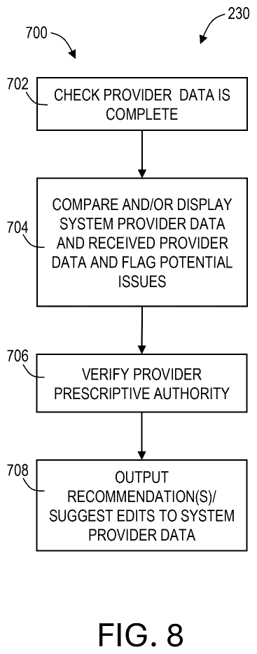

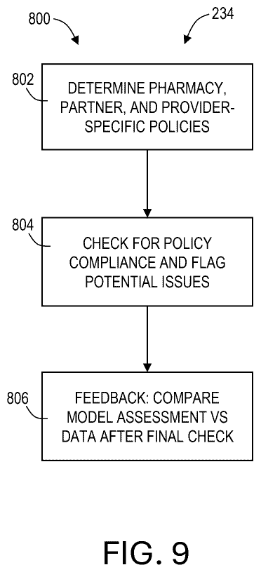

Particularly, the Decision Support System may include six main modules with the purpose of ensuring accuracy, supporting the determination of whether Clarification is required, and recommending edits or further analysis during the Prescription Verification Process: Prescription Data Validation module (FIG. 4), Clinical Prescription Safety Review module (FIG. 5), Regulatory Validation Review module (FIG. 6), Patient Validation module (FIG. 7), Provider Validation module (FIG. 8), and Pharmacy Policies & Standards Review module (FIG. 9). In some examples, the Decision Support System further includes a summarization module configured to summarize the outputs of the different software modules of the Decision Support System. For example, the summarization module may be configured to aggregate, deduplicate, and/or otherwise condense and summarize the assessment(s) and/or recommendation(s) generated by the other software modules of Decision Support System.

Each module may use or be implemented as an artificial intelligence (AI) module or algorithm. For example, each module may include or be configured to leverage and utilize one or more decision trees, predictive models, large language models (LLMs), Retrieval Augmented Generation (RAG) enhanced LLMs, rules-based engines, and/or any other suitable algorithmic and/or AI tools. For example, one or more modules may include enhanced LLM prompting using a RAG-enhanced algorithm utilizing public, private, and/or proprietary data.

Aspects of Decision Support Systems for Prescription Verification may be embodied as a computer method, computer system, or computer program product. Accordingly, aspects of the Decision Support Systems may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the Decision Support Systems may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of Decision Support Systems may be written in one or any combination of programming languages, including an object-oriented programming language (such as Java, C++, Python, Ruby, etc.), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the Decision Support Systems may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the Decision Support System. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

### Examples, Components, and Alternatives

The following sections describe selected aspects of illustrative Decision Support Systems for prescription review and fulfillment as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Prescription Fulfillment Workflow

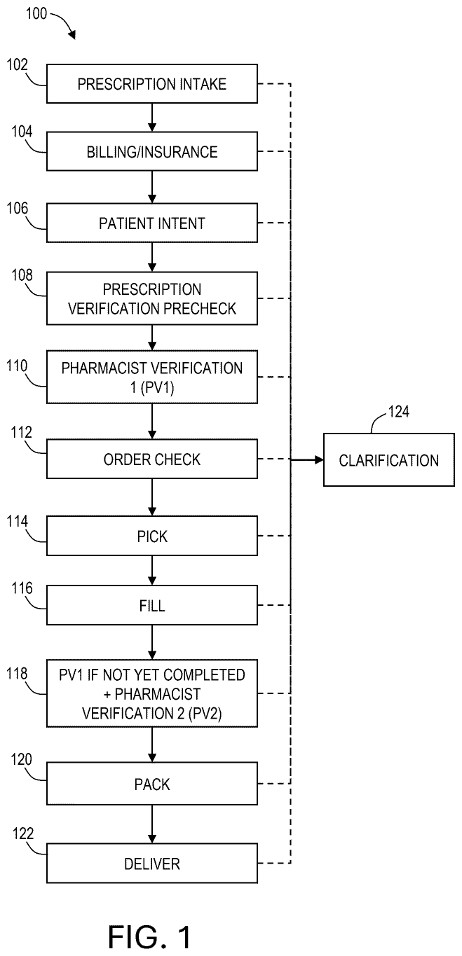

Turning to FIG. 1, an example of an overall prescription fulfillment workflow 100 is depicted for context. Aspects of Decision Support Systems described herein may be utilized in the workflow steps described below. Where appropriate, reference may be made to components and systems that may be used in carrying out each step. These references are for illustration, and are not intended to limit the possible ways of carrying out any particular step of the method.

FIG. 1 is a flowchart illustrating steps performed in an illustrative workflow, and may not recite the complete process or all steps of the workflow. Although various steps of workflow 100 are described below and depicted in FIG. 1, the steps need not necessarily all be performed, and in some cases may be performed simultaneously or in a different order than the order shown.

Step 102 of workflow 100 includes a prescription intake process wherein prescription information is received for a patient. Prescription intake may include any suitable steps configured to receive a medical prescription from a provider, physician, or clinic, and may be conducted electronically, including over the Internet. The prescription intake process ensures accurate capture of the patient and medication data. The prescription intake process may include receiving a prescription fulfillment request including one or more of patient data associated with the patient, provider data associated with the provider, physician, or clinic, and prescription data associated with the prescription (e.g., medication type, treatment plan, etc.). This step may involve the use of software to record and manage the patient data, provider data, and/or prescription data, ensuring precision and efficiency in handling the information. For example, the patient data, provider data, and/or prescription data may be transcribed or otherwise entered into a database of the Decision Support System or that is accessible by the Decision Support System.

Step 104 of workflow 100 includes confirming relevant information with relevant billing and insurance functions. For example, the prescription may be communicated to the patient's insurance company for confirmation of benefits and to receive information such as patient-specific DURs.

Step 106 of workflow 100 includes determining patient intent. For example, the pharmacy may call, email, or message the patient to determine whether the patient wishes to fill the prescription, or may be contacted by the patient for the same purpose. In general, the prescription filling process is placed on hold until patient intent is determined. Determining patient intent is a pivotal point in the workflow, as it directly influences the subsequent steps of the prescription fulfillment process. This step may involve automated systems to track and record patient responses for future reference and efficiency.

Step 108 of workflow 100 includes performing the Prescription Verification Precheck with respect to the prescription and patient in question. As explained throughout this disclosure, the Prescription Verification Precheck is a completely automated process performed by the Decision Support System. As discussed herein, the Decision Support System includes the plurality of software modules, programs, and/or algorithms configured to perform the Prescription Verification Precheck involving a comprehensive analysis of the prescription against a database of medication standards and patient history, ensuring that the prescribed medication is appropriate and safe for the patient's specific health profile. The Decision Support System may include the following software modules, programs, and/or algorithms configured to perform one or more aspects of the Prescription Verification Precheck:
Regulatory Validation module: This module takes into consideration federal and state level requirements to ensure compliance with legal and regulatory requirements. This review encompasses verifying the legality of a prescription, the prescriber's prescriptive authority, adherence to state-specific dispensing parameters, and ensuring all required prescription information is present. This critical assessment also identifies potential fraudulent prescriptions and utilizes Narx (Narcotics, Sedatives, and Stimulants) scores for controlled substances.
Patient Validation module: This module ensures that patient data for the prescription is accurate and complete. For example, system patient data which is stored in a database of the Decision Support System is compared to the patient data communicated to the system by the provider in step 102 of workflow 100. In other words, Patient Validation module may be configured to check and verify that the patient data received in the prescription fulfillment request in step 102 was properly transcribed and stored in a database accessible by Decision Support System. This includes checking and verifying that any patient allergy information is accurate or present if necessary. The Patient Validation module is further configured to verify that all required patient data (e.g., name, date of birth, contact information, etc.) is present and accessible by Decision Support System.
Provider Validation module: This module validates the provider data in a similar manner to how Patient Validation module validates the patient data, described above. For example, Provider Validation Module may ensure all required provider data is stored in the database of Decision Support System, that the system provider data stored in the database of Decision Support System matches the provider data received in the prescription fulfillment request in step 102, and that the provider has the proper prescriptive authority to prescribe the medication to the patient. For example, Provider Validation module may check and verify the provider's qualifications or licenses.
Prescription Data Validation module: This module ensures that the prescription data (e.g., SIG, NDC, etc.) is accurate, complete, and/or consistent. The prescription data may include the SIG of the prescription, the National Drug Code (NDC) of the medication, the medication name, the medication strength, the medication dosage, the medication form, the Dispense As Written (DAW) code, the written date of the prescription, the earliest fill date of the prescription, the written days' supply, the number of written refills, and/or any other suitable information associated with the medication being prescribed to the patient and the particular treatment being performed. The Prescription Validation Module is configured to verify that the system prescription data stored in the system database matches the prescription data transmitted to the system in step 102. Additionally, the Prescription Validation Module is configured to verify accuracy and consistency of the prescription data. For example, the Prescription Validation may be configured to check that the total medication quantity, the dosage quantity, and the dosage frequency of the prescription aligns with the treatment duration or how many days the patient is instructed to take the medication.
Clinical Prescription Safety module: This module is configured to perform two sets of safety checks—patient-agnostic safety checks and a patient-centric safety checks. The patient-agnostic safety checks include, for example, evaluating whether the prescribed dose falls within a safe range and identifying inherently high-risk drugs. The patient-centric safety checks extends to considering the patient's unique profile, including potential drug interactions, allergies, disease states, and whether there are redundant therapies. Doses also may be adjusted based on patient demographics (age, sex, BMI, etc.). The patient-agnostic and patient-centric safety checks are supported by robust datasets that include the latest information on drug efficacy and safety, providing an up-to-date framework for the evaluation of prescriptions.
Pharmacy Policies & Standards module: The pharmacy may have specific policy standards that must also be confirmed within a Prescription Verification process. These may include attaching the correct ancillary kit, honoring partner terms, etc. Compliance is ensured through a series of automated checks that align the pharmacy's operations with internal guidelines and partner arrangements.

Each of the above-described software modules or programs of Decision Support System may include or be configured to leverage and utilize any suitable AI models or algorithmic tools (e.g., decision trees, predictive models, LLMs, RAG-enhanced LLMs, rules-based engines, etc.) in order to perform the one or more checks or validation steps of the Prescription Verification Precheck described above. In some examples, one or more of the software modules of Decision Support System are configured to generate and output one or more assessments including one or more recommendations, prescription edits, and/or queue the prescription for Clarification based on the results of the checks or verifications performed by the software modules. For example, the software modules of the Decision Support System may be configured to identify one or more inaccuracies and determine prescription edits that fix the one or more identified inaccuracies. If the Decision Support System is unable to determine a modification or edit that fixes the one or more inaccuracies, the Decision Support System may queue the prescription for Clarification from the provider or the patient. In some examples, the Decision Support System is configured to output one or more recommendations indicating any problems detected by the software modules, recommended modifications to the prescription data, provider data, and/or patient data based on any of the detected problems, and/or whether the software modules deemed the prescription to be satisfactory and ready to fill without modification or whether the prescription requires further Clarification. In addition to the output recommendations, the Decision Support System may be configured to output any other relevant information that is gathered during the Prescription Verification Precheck. For example, the Regulatory Validation module may be configured to gather and output any relevant regulatory information (e.g., the federal and state level requirements applicable to the prescription) for review by the pharmacist.

In some examples, the software modules of the Decision Support System are implemented to leverage natural language processing, which may or may not include the use of an LLM, in order to generate and craft the output recommendations and/or other relevant information. For example, flagged aspects of the prescription, suggested edits, requests for Clarification, and/or an indication that the prescription appears to be satisfactory based on the checks performed by the Decision Support System may be highlighted on the user's screen and/or an alert or other message may be displayed to the user (e.g., a pharmacist). An LLM may be utilized to craft the explanation for any given message, preset messages may be displayed deterministically, or a combination of approaches may be used to generate the output recommendations of the Decision Support System.

In some examples, the Decision Support System is configured to perform a self-assessment to determine whether the Prescription Verification Precheck was successful and the medical prescription is in a satisfactory condition to be filled, or whether the Prescription Verification Precheck was unsuccessful and the medical prescription is in an unsatisfactory condition and is not ready to be filled. For example, if the Decision Support System did not detect any issues during the Prescription Verification Precheck or if minor issues were detected and the Decision Support System determined prescription edits that fixed the issues with a high degree of confidence, the Prescription Verification Precheck may be deemed successful by the Decision Support System and the prescription is determined to be in the satisfactory condition. In contrast, if the Decision Support System identified one or more issues that require Clarification or if the Decision Support System identified one or more issues that the Decision Support System was unable to fix by modifying the prescription data, the Prescription Verification Precheck may be deemed unsuccessful by the Decision Support System and the prescription is determined to be in unsatisfactory condition to be filled.

In some examples, if the Decision Support System determines that the Prescription Verification Precheck was unsuccessful and the prescription is in the unsatisfactory condition, step 110 of workflow 100 includes a Pharmacist Verification (PV1) performed prior to performing the order check and picking and filling the prescription. For example, if the Decision Support System identifies one or more issues with the prescription that may require Clarification or if the Decision Support System is unable to determine prescription edits that resolve the issues with a high degree of confidence, workflow 100 may include performing the PV1 in step 110 prior to filling the prescription. This ensures that any issues with the prescription data are resolved prior to filling the prescription. As discussed further below, in some examples, if the Decision Support System determines that the Prescription Verification Precheck is successful and that no identified issues remain present in the prescription, the PV1 may be postponed until after filling the prescription and performed at the same time that a Pharmacist Verification 2 (PV2) is performed.

Performing the PV1 includes a pharmacist reviewing the medical prescription for completeness, accuracy, and/or consistency. The results or assessments of the Prescription Verification Precheck performed by the Decision Support System (e.g., one or more recommendations, suggested edits, and/or flagged issues, etc.) are provided to the pharmacist for reference and to assist the pharmacist in performing the Pharmacist Verification (PV1). In step 110, the pharmacist may determine whether the prescription is in condition for proceeding to filling or whether the prescription requires Clarification at step 124, discussed further below.

Step 112 of workflow 100 includes performing an order check prior to picking and filling the prescription in steps 114 and 116. In some examples, step 112 includes awaiting full confirmation from the patient and performing a final check of the order before it proceeds to fulfillment. In this step, partially confirmed shipments wait to be fully confirmed by the patient. Fully confirmed shipments may also be blocked for technicians to perform internal quality checks.

Step 114 of workflow 100 includes picking the prescription and step 116 of workflow 100 includes filling the prescription. This may include preparing the physical prescription, such as printing a prescription label including a SIG of the prescription and any other required information and putting the required number of pills into a pill container.

Step 118 of workflow 100 includes performing the PV1 if not yet completed and performing a second Pharmacist Verification (PV2). For example, as discussed above, if the Prescription Verification Precheck performed by the Decision Support System was successful and the prescription is determined to be in the satisfactory condition to be filled, the PV1 may be performed at the same time as the PV2 in step 118 after the filling in step 116. Alternatively, if the Prescription Verification Precheck performed by the Decision Support System was unsuccessful, the PV1 is performed prior to filling the prescription in step 110. The PV1 involves the pharmacist reviewing the prescription for data accuracy, completeness, and/or consistency. The PV2 involves the pharmacist reviewing the physical filled prescription. For example, the PV1 may include checking the SIG for completeness and accuracy and the PV2 may include checking that the physical quantity of pills in the filled prescription is the correct number. The PV1 and PV2 are standardized processes that pharmacists are required to take prior to a prescription being dispensed to the patient.

Step 120 of workflow 100 includes packing the prescription and step 122 of workflow 100 includes delivering the prescription to the patient. The prescription may be delivered to the patient in person, mailed to the patient, and/or provided to the patient in any other suitable manner. In some examples, fulfillment and delivery are executed utilizing packaging and logistics solutions to ensure that medications reach patients in a timely and secure manner.

At any point in the workflow 100, the prescription may be flagged for Clarification in step 124 of workflow 100, meaning some aspect of the prescription is less than adequate to permit safe completion. For example, a person (e.g., the pharmacist) or a software application (e.g., a software module of the Decision Support System) may identify ambiguities or discrepancies in the prescription that need to be resolved. In some examples, if the Decision Support System identifies one or more discrepancies in the prescription that the Decision Support System is unable to resolve, the Decision Support System places the prescription into the Clarification Queue in step 124. If a prescription is placed into the Clarification Queue in step 124, which may happen at any of steps 102-122, but may particularly occur in steps 102, 104, 108, 110, 112, and/or 118, the prescription is reviewed by a pharmacist or other qualified entity (e.g., a technician). If an actual Clarification is warranted, then the prescribing physician, the prescribing clinic, and/or the patient is contacted to provide or correct the necessary information such that the process can continue. For example, if a potentially harmful drug interaction for the patient is identified, the prescribing clinic and/or physician may be contacted to provide Clarification. If an issue with patient allergy information is identified, the patient may be contacted directly to provide the Clarification. In some examples, after the Clarification is provided by the physician and/or the patient, the prescription may be appropriately modified to resolve the issues or discrepancies and workflow 100 may return to step 108 in which the Decision Support System performs the Prescription Verification Precheck or step 110 in which the pharmacist performs the PV1 for the now clarified prescription.

The pharmacist and other staff members carry out the various functions of workflow 100 using one or more software applications to display and edit prescription and patient information, to store and retrieve data, to provide alerts and messages, and to conduct other software functionality in support of the individual tasks. As described herein, the Decision Support System is configured to perform the Prescription Verification Precheck in step 108 using artificial intelligence modules and/or any other suitable algorithmic tools.

In each of these assessments, the Decision Support System ensures all standards are met, identifying any areas that need to be addressed before it is safe to dispense the medication. Several outcomes may be available within each check. These include obtaining a patient and/or provider Clarification, providing a soft consultation to the patient and/or provider, maintaining patient profile hygiene by eliminating duplicative prescriptions, and making or suggesting edits to the prescription.

A built-in feedback loop enables the Decision Support System to continually learn based on feedback from the overall system. When Clarifications are reviewed by pharmacists, they are each categorized as to whether the Clarification was actually necessary or not. This information funnels back into the intelligence engine as a reinforcement mechanism. Similarly, when Prescription Verification Precheck recommendations are reviewed, information on whether or not the Prescription Verification Precheck recommendation was implemented is fed back to the system.

### B. Illustrative Decision Support System and Related Algorithms

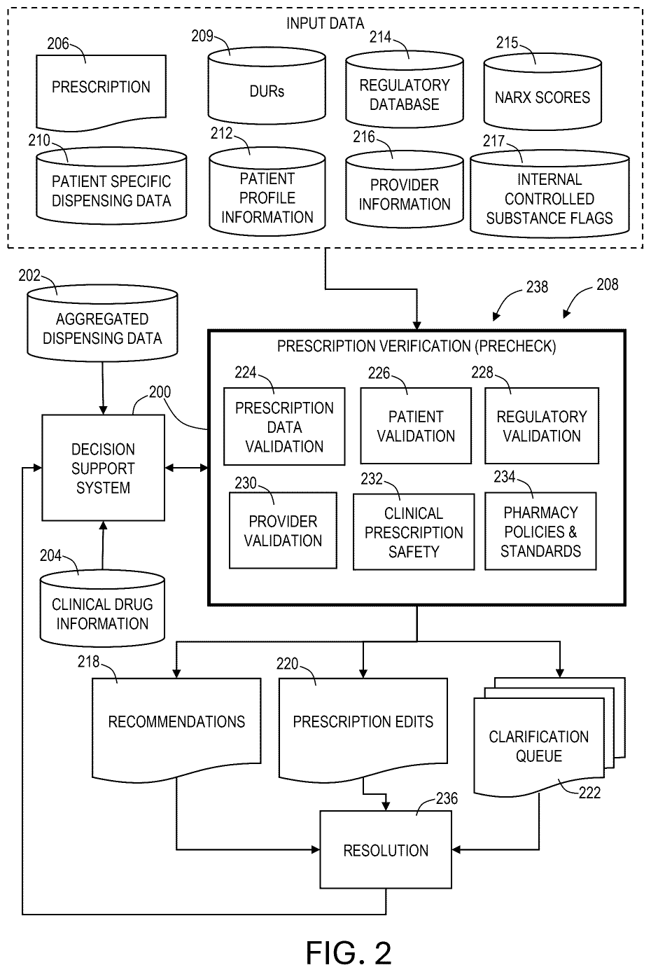

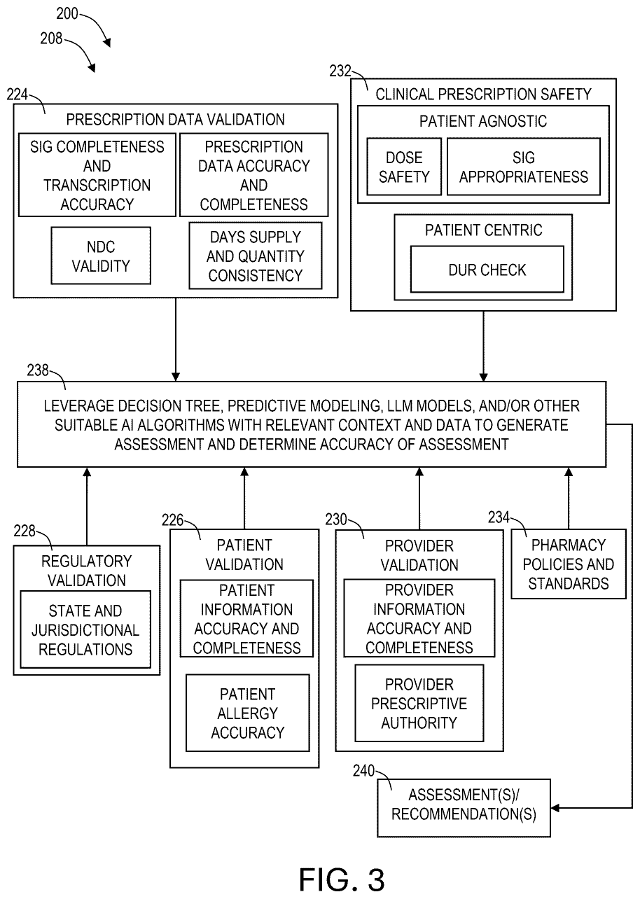

As shown in FIGS. 2-11, this section describes an illustrative Decision Support System 200 and associated algorithms. FIGS. 2 and 3 are schematic block diagrams depicting components of Decision Support System 200. FIGS. 4-11 are flow charts depicting steps of various algorithms configured to be carried out by Decision Support System 200 when performing an overall Prescription Verification Precheck 208 process or algorithm, as described in step 108 of workflow 100 above.

As shown in FIG. 2, Decision Support System 200 is a software application that takes aggregated dispensing data 202 (e.g., historical prescription dispensing data) and clinical drug information 204 into account when performing a Prescription Verification Precheck 208 in a prescription fulfillment workflow, e.g., step 108 of workflow 100 described above.

The input data utilized by Decision Support System 200 to perform the Prescription Verification Precheck 208 process includes the prescription 206, drug utilization reviews (DURs) 209 of the patient, NARX scores 215 of the medication, internal controlled substance flags 217, patient-specific dispensing data 210 (e.g., demographic information, health records of the patient, allergies, etc.), patient profile information 212 (e.g., name, date of birth, contact information), one or more regulatory databases 214 (e.g., databases storing relevant state or federal laws and regulations), provider information 216 (e.g., provider name, contact information, and prescriptive authority or licenses), and/or any other suitable input data. For example, the DURs 209 may include insurance DURs received from an insurance company of the patient and/or internal DURs generated by combining patient profile information 212, patient-specific dispensing data 210, and/or any other relevant dispensing data and utilizing one or more authoritative medical databases, such as First Databank (FDB™), to analyze the information. Decision Support System 200 is configured to assess and evaluate the input data when performing the Prescription Verification Precheck 208 and come up with one or more recommendations 218 to the user, one or more prescription edits 220 of the prescription, or Decision Support System 200 may queue the prescription for clarification 222 and flag any potential issues for the user to follow up on. The goal of Decision Support System 200 is to increase dispensing accuracy and processing efficiency.

As discussed above, Decision Support System 200 includes a plurality of software programs, modules, and/or algorithms configured to perform one or more checks, calculations, or evaluations of Prescription Verification Precheck 208. For example, performing the Prescription Verification Precheck may include validating prescription data, patient data, and/or provider data, performing a clinical prescription safety check including a patient-agnostic safety check and a patient-centric safety check, validating that the prescription is in accordance with regulatory requirements (e.g., state or federal laws and regulations), and/or validating that the prescription is in accordance with policies and standards of the pharmacy dispensing the prescription. In some examples, Decision Support System 200 includes a respective software program or module configured to perform or assist with each of the various aspects of the Prescription Verification Precheck discussed above. For example, as shown in FIGS. 2 and 3, Decision Support System 200 may include a Prescription Data Validation module 224, Patient Validation module 226, Regulatory Validation module 228, Provider Validation module 230, Clinical Prescription Safety module 232, and/or a Pharmacy Policies & Standards module 234.

In some examples, the software modules of Decision Support System 200 are implemented to leverage natural language processing, which may or may not include the use of an LLM, in order to generate and craft the output recommendations, prescription edits, and/or other relevant information. For example, flagged aspects of the prescription, any recommendation(s) 218, prescription edit(s) 220, and/or Clarifications 222, and/or an indication that the prescription appears to be satisfactory based on the checks performed by Decision Support System 200 may be highlighted on the user's screen and/or an alert or other message may be displayed to the user. An LLM may be utilized to craft the explanation for any given message, preset messages may be displayed deterministically, or a combination of approaches may be used to generate the output recommendations of Decision Support System 200.

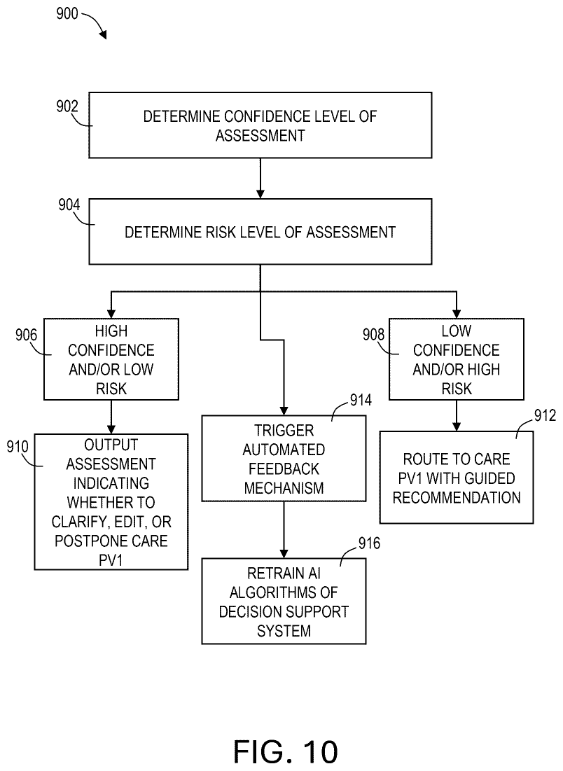

In some examples, as discussed further below with reference to FIG. 10, Decision Support System 200 includes a self-assessment algorithm 900 or self-assessment software program configured to perform a self-evaluation of the prescription assessment and the output recommendations (e.g., recommendation(s) 218, prescription edits 220, and/or Clarifications 222) of Decision Support System 200 prior to or in conjunction with displaying or otherwise outputting the recommendations to the user. For example, self-assessment algorithm 900 of Decision Support System 200 may be configured to determine a confidence level of the generated output recommendations, a risk level of the prescription assessment, and determine one or more appropriate actions based on the confidence assessment and risk level assessment.

For example, if the Prescription Verification Precheck is deemed successful by self-assessment algorithm 900 of Decision Support System 200, such as when Decision Support System 200 identifies no issues or is able to perform prescription edits that resolve any identified issues with a high degree of confidence, Decision Support System 200 determines that the PV1 (e.g., step 110 of workflow) can be postponed until after filling the prescription. In some examples, if the Prescription Verification Precheck is deemed unsuccessful by self-assessment algorithm 900 of Decision Support System 200, such as when Decision Support System 200 identifies one or more issues that may require Clarification or issues that Decision Support System 200 is unable to resolve with a high degree of confidence, Decision Support System 200 determines that the PV1 should be performed prior to filling the prescription in order to resolve the issues prior to filling the prescription.

As shown in FIG. 2, the one or more outputs of Decision Support System 200 (e.g., recommendation(s) 218, prescription edits 220, and/or Clarifications 222) may be utilized by Decision Support System 200 and/or referenced by a user (e.g., a pharmacist) to determine a resolution 236 for the prescription. Resolution 236 may include determining that the prescription is satisfactory to fill without making any modifications to the prescription, determining that Clarification is required and obtaining the required Clarification (e.g., from the provider, patient, etc.), and/or modifying one or more aspects of the prescription (e.g., the SIG) prior to filling and dispensing the prescription.

In some examples, feedback is generated based on whether resolution 236 matches or is in line with the one or more outputs of the Decision Support System 200, e.g., the proposed recommendations 218, prescription edits 220, and/or Clarifications 222 generated by Decision Support System 200 when performing Prescription Verification Precheck 208. A built-in feedback loop enables Decision Support System 200 to continually learn based on the generated feedback. For example, when the output recommendations 218, prescription edits 220, and/or Clarifications 222 are reviewed by pharmacists, the outputs may each be categorized as to whether the recommended action was accurate or actually necessary or not. This information funnels back into Decision Support System 200 as a reinforcement mechanism. Similarly, when the output recommendations 218, prescription edits 220, and/or Clarifications 222 are reviewed, information on whether or not the outputs were implemented is fed back to Decision Support System 200.

As shown in FIG. 3, each of the software modules of Decision Support System 200 includes or is configured to leverage and utilize one or more AI modules, programs, and/or algorithms 238 (AKA algorithmic tools), such as decision trees, predictive modeling, LLMs, RAG-enhanced LLMs, and/or rules-based engines configured to perform the one or more checks or validation steps of Prescription Verification Precheck 208 performed by Decision Support System 200. AI algorithms 238 and/or the software modules of Decision Support System 200 may include a Python script or other set of instructions configured to be executed by any suitable data processing system discussed herein to perform one or more aspects of Prescription Verification Precheck 208. In some examples, the software modules of Decision Support System 200 are themselves AI algorithms 238 and/or may comprise one or more algorithms (e.g., deterministic algorithms) configured to facilitate the use of AI algorithms 238 for the purpose of performing the actions of the respective software module. As shown in FIG. 3, the one or more AI algorithms 238 of Decision Support System 200 are configured to output one or more assessment(s) 240 or recommendations, which may include output recommendations 218, prescription edits 220, and/or Clarifications 222, discussed above and shown in FIG. 2.

In some examples, one or more of the software modules of Decision Support System 200 are integrated with an LLM-based algorithm to facilitate interpreting and evaluating complex medical data with precision. For example, an LLM-based algorithm (e.g., a RAG-based LLM) may be prompted with relevant context and data to assess the prescription for the criteria in question. The RAG-based LLM may cross-reference multiple authoritative data sources, such as DailyMed, to provide a thorough evaluation of the prescription. In some examples, the RAG-based LLM is integrated with a vector database allowing for a more dynamic and responsive analysis that adapts to the unique facts of each patient's profile. This approach ensures that the LLM's assessment is both comprehensive and adjusted to individual patient histories. Although vector databases are referenced here and elsewhere in the present disclosure, suitable alternatives may be utilized where appropriate. For example, the system may utilize a document database with vector search.

Each topic of the Prescription Verification Precheck may have one or more of its own tailored prompts and context, such that the appropriate assessment is performed. For example, when checking for SIG consistency, the RAG-based LLM may leverage the pharmacy's historical dispensing data to know the common SIGs and dosage routes for the medication being prescribed. The system checks that the SIG is complete, that it is consistent with the medication, that it is logically correct, and that the prescribed quantity and days of supply are sufficient to fulfill the SIG. In this context, to check for completeness the system may consider whether the Method, Quantity, Dosage, Frequency, and Route of Administration are all meaningfully present. Similarly, the system checks for consistency and logical correctness by checking, e.g., that the route of administration matches the medication prescribed; that there are no conflicting instructions; and that the SIG is grammatically correct. The LLM assesses the SIG to ensure consistency with quantity fields written on the script. For example, a SIG may read “take two 10 mg tablets daily for 14 days.” For this prescription to pass, the SIG must follow grammatical guidelines, the dosage must be 10 mg tablets, the route must be oral, with a days' supply of 14 and a quantity of 28.

With respect to other topics, such as safe dosage range and allergies, the RAG-based LLM utilizes reference data such as DailyMed and the patient's profile. In some examples, one or more of the reference data sources may be embedded in a vector database. In some examples, one or more of the reference data sources may be fully included in the prompt.

The LLM returns an answer using deterministic and/or a natural language explanation, such as “Allergy Check Passed” and “The patient has no known allergies, so there appear to be no allergy-related conflicts with the prescribed medication.” The LLM's output is presented in a clear and understandable format. The deterministic responses, coupled with natural language explanations, facilitate seamless communication between the system and the healthcare professional. This clarity is essential in ensuring that the evaluations are easily interpreted and acted upon, further enhancing the safety and efficacy of the Prescription Verification process.

As shown in FIG. 3, in some examples, the software modules of Decision Support System 200 specifically include a Prescription Data Validation module 224 (see FIG. 4), a Clinical Prescription Safety module 232 (see FIG. 5), a Regulatory Validation module 228 (see FIG. 6), a Patient Validation module 226 (see FIG. 7), a Provider Validation module 230 (see FIG. 8), and a Pharmacy Policies & Standards module 234 (see FIG. 9). As discussed above, each of the software modules of Decision Support System 200 include one or more algorithms or algorithmic tools that are configured to perform a series of checks and/or verification steps during the Prescription Verification Precheck 208. FIGS. 4-9 depict example algorithms of the software modules of Decision Support System 200. Each of the algorithms shown in FIGS. 4-9 may be implemented as any one of the AI algorithms 238 or algorithmic tools discussed above, such as LLM-based algorithms, rules-based engines, and/or decision trees to check each prescription for errors and flag instances of potential noncompliance.

FIG. 4 depicts an example Prescription Data Validation algorithm 300 of Prescription Data Validation Module 224 of Decision Support System 200, discussed above. Each step of Prescription Data Validation algorithm 300 may be implemented as rules-based engines or decision trees to check the prescription data for errors or inconsistencies. As shown in FIG. 4, Prescription Data Validation algorithm 300 includes verifying SIG completeness, SIG transcription, and SIG consistency in step 302, verifying prescription data accuracy and completeness in step 304, verifying NDC validity in step 306, verifying medication days' supply and quantity consistency in step 308, and outputting one or more recommendations or suggested edits in step 310 based on the analysis performed in steps 302, 304, 306, and/or 308 of Prescription Data Validation algorithm 300.

In some examples, verifying SIG completeness and SIG transcription accuracy in step 302 of Prescription Data Validation algorithm 300 includes checking to ensure that the SIG includes all required information fields, such as a dosage, a dose frequency, a route of administration, a duration of treatment, and/or a total medication quantity. In some examples, step 302 includes checking consistency between the total medication quantity, the dose quantity, the dose frequency, and the duration of treatment. For example, a SIG may read “take two 10 mg tablets daily for 14 days.” For this prescription to pass, the SIG must follow grammatical guidelines, the dosage must be 10 mg tablets, the route must be oral, with a days' supply of 14 and a quantity of 28. In some examples, step 302 of algorithm 300 includes comparing system SIG data stored in a database of Decision Support System 200 to the original SIG data received in a prescription fill request from the provider and ensuring that the system SIG data matches the SIG data from the prescription fill request.

In some examples, verifying prescription data accuracy and completeness in step 304 of Prescription Data Validation algorithm 300 includes checking to ensure that all required prescription data fields are filled, such as drug name, drug strength, drug dosage form, written quantity, date written, and/or a diagnosis code. In some examples, step 304 includes comparing system prescription data stored in a database of Decision Support System 200 to the original prescription data received in a prescription fill request from the provider and ensuring that the system prescription data matches the prescription data originally provided in the prescription fill request.

In some examples, verifying NDC validity in step 306 of Prescription Data Validation algorithm 300 includes determining whether the prescription is to be filled for a brand-name drug or if an equivalent generic drug is available that may be used instead. For example, the prescription data may include an NDC code for the medication and step 306 may include determining whether the NDC code refers to a brand-name drug or a generic drug. If the NDC code refers to a brand-name drug, step 306 may include checking the Dispense As Written (DAW) code for the prescription, which may be included in the prescription data received in the prescription fulfillment request, to determine whether switching to an equivalent generic drug is permitted. If the DAW code indicates that switching to an equivalent generic drug is permitted, step 306 of algorithm 300 may include determining whether an equivalent generic drug exists and switching the prescription to be filled using the generic version of the drug. If the NDC code is originally for a generic version of the drug, step 306 may include verifying accuracy of the NDC code and proceeding with using the generic version of the drug.

In some examples, verifying medication days' supply and quantity consistency in step 308 of Prescription Data Validation algorithm 300 includes verifying that the prescribed duration of treatment or days' supply is consistent with the medication quantity of the prescription. This may be determined based on the dose quantity and dose frequency. Additionally, step 308 may include checking the prescribed days' supply and quantity received in the prescription fill request matches the written or system days' supply and quantity of the prescription being filled stored in the database of Decision Support System 200.

In some examples, step 310 of Prescription Data Validation algorithm 300 includes outputting one or more recommendations or suggested edits. For example, in response to identifying an inconsistency in the SIG in step 302, step 310 may include outputting an alert to the pharmacist indicating the inconsistency and/or a proposed edit of the SIG that is determined to fix the inconsistency. As discussed herein, step 310 of algorithm 300 may include the use of natural language processing models, such as an LLM, to craft and generate the output recommendations and/or edits.

FIG. 5 depicts an example Clinical Prescription Safety algorithm 400 of Clinical Prescription Safety module 232 of Decision Support System 200. As shown in FIG. 5, Clinical Prescription Safety algorithm 400 includes performing a patient-agnostic safety check in step 402, performing a patient-centric safety check in step 408, and outputting one or more recommendations based on the results of the patient-agnostic and patient-centric safety checks in step 412.

As shown in FIG. 5, in some examples, performing the patient-agnostic safety check in step 402 of Clinical Prescription Safety algorithm 400 includes a step 404 of checking dose safety for the medication of the prescription and/or a step 406 of checking SIG appropriateness for the medication. Checking the dose safety and SIG appropriateness in steps 404 and 406 may include referencing one or more authoritative medical databases and/or historical dispensing data to determine a safe dosage range for the particular medication and checking that the dosage in the SIG of the prescription falls within the safe dosage range. If the dosage in the SIG of the prescription exceeds or otherwise falls outside of the safe dosage range for the medication, performing the patient-agnostic safety check in step 402 may further include checking for any notes received from the provider that may indicate a justification for the dosage being outside of the recommended range. If no indication is found explaining why the dosage falls outside of the recommended dosage range, the prescription fails the patient-agnostic safety check and may be flagged for Clarification. In such examples, the output recommendation output in step 412 Clinical Prescription Safety algorithm 400 may include a notification indicating that the dosage is outside of the safe dosage range. Step 412 may further include placing the prescription in the Clarification queue with other prescriptions requiring Clarification. In such examples, Clarification may be requested from the provider explaining why the dosage of the prescription exceeds the safe or recommended dosage range for the medication. If the dosage in the SIG of the prescription falls within the safe dosage range, the prescription may pass the patient-agnostic safety check, and the output recommendation may indicate that the prescription passed the patient-agnostic safety check and/or that the dosage falls within the safe dosage range.

As shown in FIG. 5, in some examples, performing the patient-centric safety check in step 408 of Clinical Prescription Safety algorithm 400 includes performing a drug utilization review (DUR) of the patient in step 410. In some examples, performing the DUR includes reviewing patient data to identify one or more of a history of clinical drug abuse by the patient, drug-disease contraindications, adverse drug interactions, and/or any other drug precautions based on age, allergies, gender, and/or any other variables specific to the patient. If no problems are detected when reviewing the patient data in view of the medication being prescribed, the prescription may pass the patient-centric safety check and in step 412 the output recommendation may indicate that the prescription passed the patient-centric safety check and/or that no problems were detected when performing the DUR of the patient. If one or more issues are detected when performing the DUR, the prescription may fail to pass the patient-centric safety check and in step 412 the output recommendation may indicate the one or more detected issues, recommend Clarification from the provider or patient, and/or otherwise indicate that the prescription failed the patient-centric safety check.

FIG. 6 depicts an example Regulatory Validation algorithm 500 of Regulatory Validation module 228 of Decision Support System 200. As shown in FIG. 6, Regulatory Validation algorithm 500 includes determining the state or jurisdiction the prescription is under in step 502, assessing the prescription for applicable jurisdictional regulations in step 504, and displaying or otherwise outputting applicable regulations in step 506. For example, Regulatory Validation algorithm 500 may include searching one or more regulatory databases to identify laws or regulations that apply to the particular prescription being dispensed and outputting the identified laws and regulations to the pharmacist in order to aid the pharmacist in performing the Pharmacist Verification (PV1).

In some examples, in addition to identifying and outputting the applicable laws and regulations, step 504 of Regulatory Validation algorithm 500 further includes assessing the prescription to determine whether the prescription is in compliance with the applicable laws and regulations. For example, step 504 of Regulatory Validation algorithm 500 may include verifying the legality of the prescription, the prescriber's prescriptive authority, adherence to state-specific dispensing parameters, and/or ensuring all required prescription information is present.

As an example scenario, the medication prescription may be issued for a 30 day supply, but the medication prescription being dispensed is for a 90 day supply. In such examples, step 504 of Regulatory Validation algorithm 500 may include determining whether dispensing a 90 days supply for the drug is allowed per state regulations. As another example, step 504 of Regulatory Validation algorithm may include determining whether changing the drug form (e.g., pill, liquid, etc.) is allowed per state regulations for the particular drug in question. As another example, step 504 of Regulatory Validation algorithm may include determining whether substituting a particular brand-name drug for a generic version of the drug is allowed per state regulations. In some examples, step 504 of Regulatory Validation algorithm 500 includes determining whether state regulations require patient allergy input prior to dispensing the drug to the patient and determining whether the required patient allergy input has been received or must be requested prior to dispensing the prescription.

In some examples, in addition to outputting the applicable laws and regulations themselves in step 506 of Regulatory Validation algorithm 500, step 506 further includes outputting whether the prescription is determined to be in compliance with the applicable laws and regulations or not. If not, step 506 may include outputting the specific laws and regulations that the prescription is not in compliance with.

FIG. 7 depicts an example Patient Validation algorithm 600 of Patient Validation module 226 of Decision Support System 200. As shown in FIG. 7, Patient Validation algorithm 600 includes checking patient data for completeness in step 602, comparing and/or displaying system patient data and received patient data in step 604, checking patient allergy accuracy in step 606, and outputting one or more recommendations and/or suggested edits to the system patient data in step 608 in response to detecting one or more issues.

Step 602 of Patient Validation algorithm 600 may include checking that all required patient information, such as demographic information (e.g., age, gender, etc.), allergy information, and/or contact information (e.g., name, address, phone number, email, etc.) has been provided and is accessible by Decision Support System 200.

Step 604 of Patient Validation algorithm 600 includes verifying that the patient information has been transcribed into the system properly from the prescription fill request received from the provider. For example, step 604 may include Decision Support System 200 comparing the system patient data to received patient data or displaying or otherwise outputting the system patient data and received patient data next to each other on a display for review by the pharmacist. System patient data refers to data stored in a system database of Decision Support System 200 and received patient data refers to the patient data that was originally received and transmitted from the provider in the prescription fulfillment request.

In some examples, Patient Validation algorithm 600 includes step 606, which includes ensuring that patient allergy information stored in Decision Support System 200 matches patient allergy information provided by the provider, patient allergy information provided by an insurance company of the patient, and/or patient allergy information provided by the patient themselves. Step 606 may further include determining whether required patient allergy information has been provided and accounted for. For example, one or more regulations may require the patient or provider to provide their medication allergies prior to dispensing specific medications to the patient and step 606 includes ensuring that the required information has been provided.

Step 608 of Patient Validation algorithm 600 includes outputting one or more recommendations or suggested edits of the system patient data. For example, if the patient data is determined to be incomplete in step 602, step 608 may include outputting a recommendation to request the information from the provider and/or the patient. If a discrepancy is identified between the system patient data and received patient data in step 604 and/or between the system allergy information and received allergy information provided by the provider, patient, or insurance company in step 606, step 608 may include outputting one or more recommended edits of the system patient data and/or allergy information to match the received patient data or received allergy information.

FIG. 8 depicts an example Provider Validation algorithm 700 of Provider Validation module 230 of Decision Support System 200. As shown in FIG. 8, Provider Validation algorithm 700 includes checking provider data for completeness in step 702, comparing and/or displaying system provider data and received provider data in step 704, verifying provider prescriptive authority in step 706, and outputting one or more recommendations and/or suggested edits to the system provider data in step 708 in response to detecting one or more issues.

Step 702 of Provider Validation algorithm 700 includes verifying that all required provider data has been provided to Decision Support System 200 and is available for review. For example, the provider data may include one or more of a provider name, phone number, provider licenses, certifications, or identification numbers (e.g., provider NPI or DEA), and/or a supervising physician of the provider. If any required provider data is missing, step 708 of Provider Validation algorithm 700 may include outputting a recommendation to request the missing information from the provider.

Step 704 of Provider Validation algorithm 700 includes comparing the system provider data stored in the system database accessible by Decision Support System 200 to the received provider data received in the prescription fulfillment request from the provider. If the system provider data does not match the received provider data received in the prescription fulfillment request, step 708 of Provider Validation algorithm 700 may include outputting one or more suggested edits to the provider data to ensure that the provider data is accurate and matches the received provider data.

Step 706 of Provider Validation algorithm 700 includes verifying the prescriptive authority of the provider, which may include ensuring that the provider has the required certifications and/or licenses to prescribe the prescription to the patient. If the provider data indicates that the provider does not have the required authority, step 708 of Provider Validation algorithm 700 may include outputting a recommendation to request Clarification from the provider.

FIG. 9 depicts an example Pharmacy Policies & Standards algorithm 800 of Pharmacy Policies & Standards module 234 of Decision Support System 200. As shown in FIG. 9, Pharmacy Policies & Standards algorithm 800 includes determining pharmacy, partner, and/or provider-specific policies in step 802, checking for policy compliance of the prescription and flagging potential issues in step 804, and a feedback step 806 of comparing the model assessment to data after final check or review by the pharmacist. Pharmacy Policies & Standards algorithm 800 is configured to review the prescription to ensure compliance with pharmacy-specific policies and standards. For example, complying with the pharmacy-specific policies and regulations may include attaching the correct ancillary kit, honoring partner terms, etc.

If it is determined in step 804 of Pharmacy Policies & Standards algorithm 800 that the prescription does not comply with the pharmacy-specific policies and standards, Pharmacy Policies & Standards algorithm 800 may generate an output indicating the identified problems to the pharmacist. The pharmacist may then review the output of Pharmacy Policies & Standards algorithm 800 and determine whether any actions are required. In step 806 of Pharmacy Policies & Standards algorithm 800, the output of the Pharmacy Policies & Standards algorithm 800 is compared to the actions (if any) taken by the pharmacist to determine whether the output of the Pharmacy Policies & Standards algorithm 800 was accurate and/or necessary. For example, Pharmacy Policies & Standards algorithm 800 may generate an output indicating that a specific aspect of the prescription was not in accordance with the pharmacy-specific policies and standards and that an action is required, but the pharmacist may determine that this output was inaccurate and that no action was actually required. In such examples, feedback may be provided to Decision Support System 200 that Pharmacy Policies & Standards algorithm 800 generated an erroneous output that did not require any action. Decision Support System 200 may be configured to incorporate the feedback to retrain the Pharmacy Policies & Standards algorithm 800.

As shown in FIG. 10, in some examples, Decision Support System 200 includes a self-assessment algorithm 900 configured to determine the accuracy of the generated output assessments (e.g., the recommendations, flagged issues, prescription edits, requests for Clarification, etc.) that are output by the software modules of Decision Support System 200, as discussed above with reference to FIGS. 2-9. For example, self-assessment algorithm 900 may determine the accuracy of the generated output assessments as a confidence level, an accuracy score, or a probability of correctness. Self-assessment algorithm 900 may be configured to determine the accuracy of the generated output assessments pharmacist to determine appropriate action based on the confidence level and/or risk level of the assessment.

For example, step 902 of self-assessment algorithm 900 may include determining a confidence level of the assessment(s) generated by one or more of the software modules of Decision Support System 200 when performing the Prescription Verification Precheck process 208. This may include categorizing the generated assessment(s) as high-confidence assessment(s) or low-confidence assessment(s). In some examples, the confidence level for the particular assessment is determined based on prior historical assessment data, which may indicate an accuracy of previous assessments performed for the particular patient or medication in question. In some examples, the Prescription Verification Precheck is determined to be successful if the generated assessments are determined to be high confidence assessments and the Prescription Verification Precheck is determined to be unsuccessful if the generated assessments are determined to be low confidence assessments.

Explained in other words, if Decision Support System 200 identifies no issues with the prescription with a high degree of confidence and/or is able to determine prescription edits that resolve any identified issues with a high degree of confidence, the Prescription Verification Precheck may be deemed successful. In such examples, Decision Support System 200 determines with a high degree of confidence that the prescription is in a satisfactory condition to be filled without further modification. In contrast, if Decision Support System 200 identifies one or more issues requiring Clarification or any issues that Decision Support System 200 is unable to resolve with high confidence, Decision Support System 200 determines that further review or Clarification is required prior to filling the prescription and that the Prescription Verification Precheck was unsuccessful. In such examples, the Decision Support System 200 determines that the prescription is in an unsatisfactory condition to be filled without first being reviewed by the pharmacist.

In some examples, if the assessment is determined to be a high confidence assessment and/or a low confidence assessment, self-assessment algorithm 900 is configured to move forward to step 904. In some examples, if the assessment is determined to be a low-confidence level assessment, one or more other techniques may be utilized to collect multiple assessments. For example, the one or more software modules of Decision Support System 200 may be rerun N times to ensure the assessment is consistent, e.g., taking the most frequent response. In some examples in which the software modules of Decision Support System 200 are implemented with an LLM-based model, the LLM-based model may be reprompted with different linguistics and/or differing algorithms to attempt to generate a different output assessment that has a high-confidence level. In some examples, an output assessment of Decision Support System 200 may be deemed to be a non-compliant assessment, in which case the assessment output by Decision Support System 200 may be ignored for this medication and/or patient.

Step 904 of self-assessment algorithm 900 includes determining a risk level of the generated output assessment(s). Determining the risk level of the generated assessment(s) may include categorizing the assessment(s) as low risk in step 906 of algorithm 900 or high risk in step 908 of algorithm 900. The risk level of assessment(s) 240 may be determined by Decision Support System 200 based on the patient health risk, cost risk, and/or any other suitable factors of the assessment or particular prescription in question. For example, if Clinical Prescription Safety module 232 of Decision Support System 200 identifies a potentially dangerous drug interaction between the prescription and a different medication taken by the patient, the generated output assessment(s) may include a recommendation for Clarification from the provider or pharmacist, an alert to the user identifying the conflict, and/or the assessment may be categorized as a high-risk assessment by self-assessment algorithm 900. Additionally, the assessment(s) generated by Decision Support System 200 for prescriptions that are of high importance to the patient's health and/or for patient's that have severe health conditions may be automatically flagged as high-risk assessments. In contrast, if the prescription in question is unlikely to cause serious medical implications for the patient, the assessment for the particular prescription may be categorized as a low-risk assessment.

In some examples, if the generated assessments are categorized as low risk, step 910 of algorithm 900 includes outputting the assessment to the user, e.g., displaying the assessment on a user interface. For example, step 910 of algorithm 900 may include displaying one or more output recommendations to the user, such as, one or more of the recommendations 218, prescription edits 220, and/or Clarifications 222, discussed above. In some examples, Decision Support System 200 utilizes an LLM to generate and output the one or more output recommendations, which may then be displayed on a user interface to the user.

In some examples, if the generated assessments are categorized as high confidence and low risk assessments (e.g., the Prescription Verification Precheck is deemed successful), step 910 of self-assessment algorithm 900 includes Decision Support System 200 postponing the initial PV1 (e.g., step 110 of method 100) performed by the pharmacist until after the prescription has been filled. For example, if Decision Support System 200 identifies no problems with the prescription when performing the Prescription Verification Precheck or is able to resolve any identified problems (e.g., by making one or more prescription edits) with a high degree of confidence, Decision Support System 200 may facilitate postponing the PV1 until after filling the prescription. In such examples, Decision Support System 200 may queue the prescription in a list of prescriptions that are ready to be picked and filled, without first routing the prescription to first be reviewed by the Pharmacist in the first Pharmacist Verification (PV1) step. In such examples, the pharmacist performs both the PV1 and the PV2 after the prescription has been filled, but prior to the prescription being packed and delivered to the patient. Thus, in examples in which Decision Support System 200 has a high degree of confidence that the prescription contains no errors or discrepancies after performance of the Prescription Verification Precheck, the prescription may proceed directly to being filled prior to review by a pharmacist, increasing operational efficiency in the prescription fulfillment workflow.

In some examples, if assessment(s) 240 are categorized as high risk and/or low confidence assessments (e.g., the Prescription Verification Precheck is deemed unsuccessful), self-assessment algorithm 900 includes step 912, which includes Decision Support System 200 routing the prescription to PV1 in step 110 of workflow 100 described above. For example, this may include queuing the prescription in a list of prescriptions that are to be reviewed by the pharmacist in PV1 prior to filling the prescriptions. In such examples, Decision Support System 200 may include with the prescription the assessment that was generated by Decision Support System 200 when performing the Prescription Verification Precheck. For example, the prescription for review by the pharmacist may be accompanied by the generated assessment including one or more guided recommendations for the pharmacist, such as one or more recommendations 218, proposed prescription edits 220, and/or Clarifications 222. The pharmacist may then utilize and/or reference the recommendations 218, proposed prescription edits 220, and/or Clarifications 222 to assist the pharmacist in performing the PV1.

Step 912 of self-assessment algorithm 900 may occur when the assessments and/or the prescription are determined to be high risk by Decision Support System 200, when the assessments are determined to be low confidence assessments, and/or when the assessments are determined to be both high risk and low confidence assessments. For example, if Decision Support System 200 identifies one or more issues with the prescription and is unable to determine with a high degree of confidence one or more prescription edits that fix the identified issue(s), Decision Support System 200 may route the prescription to the PV1 step prior to filling the prescription. This ensures that the prescription is not filled incorrectly, which would require a large degree of rework to resolve. In some examples, Decision Support System 200 outputs in step 912 any prescription edits 220 or guided recommendations 218 for the pharmacist, even if the prescription edits 220 or guided recommendations 218 are not determined to be high confidence assessments by Decision Support System 200. The pharmacist may then perform the PV1 by reviewing the prescription for data accuracy assisted by the assessments output by Decision Support System 200.

In some examples, step 914 of self-assessment algorithm 900 includes triggering an automated feedback mechanism. Step 914 may be performed after the pharmacist has reviewed the prescription and the assessment output by Decision Support System 200 and determined a resolution of the prescription. As discussed above, feedback may be generated based on whether the assessments generated by Decision Support System 200 (e.g., output recommendations 218, prescription edits 220, and/or Clarifications 222) are determined to be accurate and/or helpful by the pharmacist or pharmacist. Additionally, feedback may be generated indicating whether the output assessment was determined to be a high-confidence or low-confidence assessment in step 902 of self-assessment algorithm 900. In some examples, when the output recommendations 218, prescription edits 220, and/or Clarifications 222 are reviewed by the pharmacist, they may each be categorized as to whether the outputs are accurate or actually necessary or not. Information including any recommended actions output by Decision Support System 200 and the actual actions performed by the pharmacist and whether they matched may be utilized to retrain one or more of the software modules and/or AI algorithms 238 of Decision Support System 200 in step 916 of self-assessment algorithm 900. In this manner, the AI algorithms 238 of Decision Support System 200 can be continually improved by continually learning based on the generated feedback.

### C. Machine Learning Example

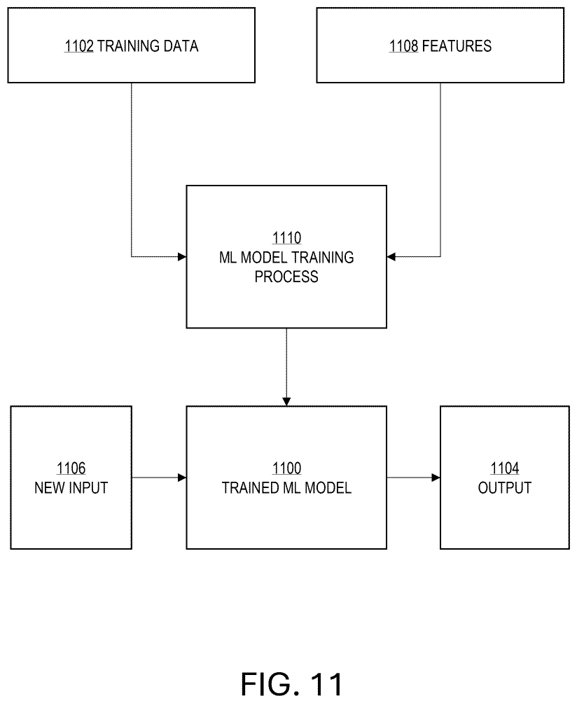

FIG. 11 depicts the training and use of an illustrative machine learning algorithm or model 1100. As mentioned above, machine learning algorithms may be utilized in one or more aspects of the Decision Support Systems and/or other systems described herein.

In general, machine learning (ML) models (also referred to as ML algorithms, ML tools, or ML programs) may be utilized to generate predictions or decisions that are useful in themselves and/or in the service of a more comprehensive program. ML algorithms “learn” by example, based on existing sample data, and generate a trained model. Using the trained model, predictions or decisions can then be made regarding new data without explicit programming. Machine learning therefore involves algorithms or tools that learn from existing data and make predictions or inferences about novel data.

Training data 1102 (e.g., labeled training data) is utilized to build trained ML model 1100, such that the ML model can produce a desired output 1104 when presented with new data 1106. In general, the ML model uses labeled training data 1102, which includes values for the input variables and values for the known correct outputs, to ascertain relationships and correlations between variables or features 1108 to produce an algorithm mapping the input values to the outputs.

Supervised learning methods may be utilized for the purposes of producing classification or regression algorithms. Classification algorithms are typically used in situations where the goal is categorization (e.g., whether a photo contains a cat or a dog). Regression algorithms are typically used in situations where the goal is a numerical value (e.g., the market value of a house).

Features 1108 may include any suitable characteristics capable of being measured and configured to provide some level of information regarding the input scenario, situation, or phenomenon. For example, if the goal is to provide an output relating to the market value of a house, then the features may include variables such as square footage, postal code, year built, lot size, number of bedrooms, etc. Although these example features are numeric, other feature types may be included, such as strings, Boolean values, etc.

Different ML techniques may be used, depending on the application. For example, artificial neural networks, decision trees, support-vector machines, regression analysis, Bayesian networks, genetic algorithms, random forests, and/or the like may be utilized to produce the trained ML model.

Trained ML model 1100 is produced by training process 1110 based on identified features 1108 and training data 1102. Trained ML model 1100 can then be utilized to predict a category or infer an output value 1104 based on new data 1106.

Large Language Models (LLMs) are a subset of machine learning that focus on understanding and generating human language. They are built using deep learning techniques, particularly neural networks and a transformer architecture, and are trained on large amounts of text or other tokenizable data to learn a wide array of language patterns and knowledge. Transformers allow the model to focus on different parts of the input text, understanding context and relationships between words.

The input text is broken down into smaller units known as tokens (words or portions of words). The model processes these tokens (represented numerically) to predict the next token in a sequence, enabling it to generate coherent and contextually relevant text. Once trained, LLMs can respond to a prompt input by generating text, which may equate to answering questions, translating languages, summarizing content, etc. Although the present description refers here to text, any tokenizable input can be used to train an LLM, such as audio and video data.

With respect to the present disclosure, ML models may be used at various points in the data processing algorithm(s). For example, one or more LLMs may be utilized in the Decision Support System to analyze, summarize, and/or synthesize information. Such an LLM may be enhanced or implemented using Retrieval Augmented Generation (RAG), in which specific data sources are leveraged to supplement the LLM's prompt and ensure more focused or informed answers. For example, RAG may include splitting a particular data source into chunks or snippets of a selected size. These chunks are then converted into embeddings, which are vector representations of the words, wherein the vector captures the meaning of that chunk of words. Functions for easily creating the embeddings are made possible by LLMs, as an LLM by definition understands word relationships and meanings, and represents such information numerically using large vectors. The vectors and text chunks are saved in a searchable vector database.

A script or other application can then receive a prompt or question, convert the prompt using the same embedding function, and search the vector database to retrieve the top N closest matches. These closest matching text chunks are combined with the original prompt to create a modified prompt that contains relevant data. The modified prompt is sent to the LLM and the answer is returned. To assist in the reduction of hallucination errors, deterministic quoting may be utilized to return summarized output information.

ML models, LLM (e.g., including RAG) and otherwise, may be utilized in the Decision Support Systems described herein. For example, see FIGS. 2-10 and accompanying description.

### D. Illustrative Data Processing System

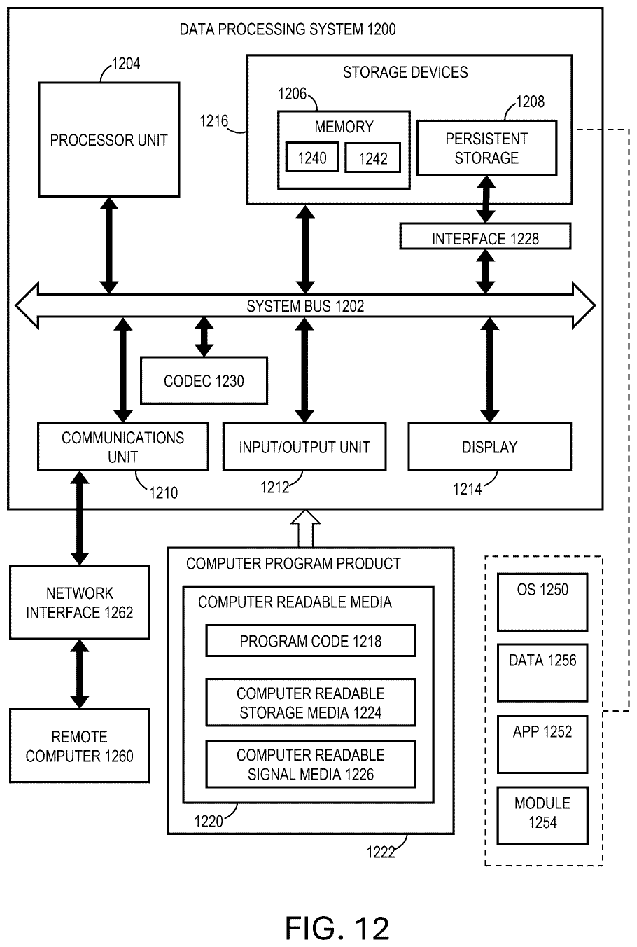

As shown in FIG. 12, this example describes a data processing system 1200 (also referred to as a computer, computing system, and/or computer system) in accordance with aspects of the present disclosure. In this example, data processing system 1200 is an illustrative data processing system suitable for implementing aspects of the pharmacy management systems and Decision Support Systems described above. More specifically, in some examples, devices that are embodiments of data processing systems (e.g., smartphones, tablets, personal computers) may be utilized for programming, using, and maintaining the Decision Support System, e.g., via a graphical user interface of one or more such devices. Moreover, these devices may be networked together (see section D).

In this illustrative example, data processing system 1200 includes a system bus 1202 (also referred to as communications framework). System bus 1202 may provide communications between a processor unit 1204 (also referred to as a processor or processors), a memory 1206, a persistent storage 1208, a communications unit 1210, an input/output (I/O) unit 1212, a codec 1230, and/or a display 1214. Memory 1206, persistent storage 1208, communications unit 1210, input/output (I/O) unit 1212, display 1214, and codec 1230 are examples of resources that may be accessible by processor unit 1204 via system bus 1202.

Processor unit 1204 serves to run instructions that may be loaded into memory 1206. Processor unit 1204 may comprise a number of processors, a multi-processor core, and/or a particular type of processor or processors (e.g., a central processing unit (CPU), graphics processing unit (GPU), etc.), depending on the particular implementation. Further, processor unit 1204 may be implemented using a number of heterogeneous processor systems in which a main processor is present with secondary processors on a single chip. As another illustrative example, processor unit 1204 may be a symmetric multi-processor system containing multiple processors of the same type.

Memory 1206 and persistent storage 1208 are examples of storage devices 1216. A storage device may include any suitable hardware capable of storing information (e.g., digital information), such as data, program code in functional form, and/or other suitable information, either on a temporary basis or a permanent basis.

Storage devices 1216 also may be referred to as computer-readable storage devices or computer-readable media. Memory 1206 may include a volatile storage memory 1240 and a non-volatile memory 1242. In some examples, a basic input/output system (BIOS), containing the basic routines to transfer information between elements within the data processing system 1200, such as during start-up, may be stored in non-volatile memory 1242. Persistent storage 1208 may take various forms, depending on the particular implementation.

Persistent storage 1208 may contain one or more components or devices. For example, persistent storage 1208 may include one or more devices such as a magnetic disk drive (also referred to as a hard disk drive or HDD), solid state disk (SSD), floppy disk drive, tape drive, Jaz drive, Zip drive, flash memory card, memory stick, and/or the like, or any combination of these. One or more of these devices may be removable and/or portable, e.g., a removable hard drive. Persistent storage 1208 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 1208 to system bus 1202, a removable or non-removable interface is typically used, such as interface 1228.

Input/output (I/O) unit 1212 allows for input and output of data with other devices that may be connected to data processing system 1200 (i.e., input devices and output devices). For example, an input device may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 1204 through system bus 1202 via interface port(s). Suitable interface port(s) may include, for example, a serial port, a parallel port, a game port, and/or a universal serial bus (USB).

One or more output devices may use some of the same types of ports, and in some cases the same actual ports, as the input device(s). For example, a USB port may be used to provide input to data processing system 1200 and to output information from data processing system 1200 to an output device. One or more output adapters may be provided for certain output devices (e.g., monitors, speakers, and printers, among others) which require special adapters. Suitable output adapters may include, e.g. video and sound cards that provide a means of connection between the output device and system bus 1202. Other devices and/or systems of devices may provide both input and output capabilities, such as remote computer(s) 1260. Display 1214 may include any suitable human-machine interface or other mechanism configured to display information to a user, e.g., a CRT, LED, or LCD monitor or screen, etc.

Communications unit 1210 refers to any suitable hardware and/or software employed to provide for communications with other data processing systems or devices. While communication unit 1210 is shown inside data processing system 1200, it may in some examples be at least partially external to data processing system 1200. Communications unit 1210 may include internal and external technologies, e.g., modems (including regular telephone grade modems, cable modems, and DSL modems), ISDN adapters, and/or wired and wireless Ethernet cards, hubs, routers, etc. Data processing system 1200 may operate in a networked environment, using logical connections to one or more remote computers 1260. A remote computer(s) 1260 may include a personal computer (PC), a server, a router, a network PC, a workstation, a microprocessor-based appliance, a peer device, a smart phone, a tablet, another network note, and/or the like. Remote computer(s) 1260 typically include many of the elements described relative to data processing system 1200. Remote computer(s) 1260 may be logically connected to data processing system 1200 through a network interface 1262 which is connected to data processing system 1200 via communications unit 1210. Network interface 1262 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 1230 may include an encoder, a decoder, or both, comprising hardware, software, or a combination of hardware and software. Codec 1230 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 1230 is depicted as a separate component, codec 1230 may be contained or implemented in memory, e.g., non-volatile memory 1242.

Non-volatile memory 1242 may include read only memory (ROM), programmable ROM (PROM), electrically programmable ROM (EPROM), electrically erasable programmable ROM (EEPROM), flash memory, and/or the like, or any combination of these. Volatile memory 1240 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 1216, which are in communication with processor unit 1204 through system bus 1202. In these illustrative examples, the instructions are in a functional form in persistent storage 1208. These instructions may be loaded into memory 1206 for execution by processor unit 1204. Processes of one or more embodiments of the present disclosure may be performed by processor unit 1204 using computer-implemented instructions, which may be located in a memory, such as memory 1206.

These instructions are referred to as program instructions, program code, computer usable program code, or computer-readable program code executed by a processor in processor unit 1204. The program code in the different embodiments may be embodied on different physical or computer-readable storage media, such as memory 1206 or persistent storage 1208. Program code 1218 may be located in a functional form on computer-readable media 1220 that is selectively removable and may be loaded onto or transferred to data processing system 1200 for execution by processor unit 1204. Program code 1218 and computer-readable media 1220 form computer program product 1222 in these examples. In one example, computer-readable media 1220 may comprise computer-readable storage media 1224 or computer-readable signal media 1226.

Computer-readable storage media 1224 may include, for example, an optical or magnetic disk that is inserted or placed into a drive or other device that is part of persistent storage 1208 for transfer onto a storage device, such as a hard drive, that is part of persistent storage 1208. Computer-readable storage media 1224 also may take the form of a persistent storage, such as a hard drive, a thumb drive, or a flash memory, that is connected to data processing system 1200. In some instances, computer-readable storage media 1224 may not be removable from data processing system 1200.

In these examples, computer-readable storage media 1224 is a non-transitory, physical or tangible storage device used to store program code 1218 rather than a medium that propagates or transmits program code 1218. Computer-readable storage media 1224 is also referred to as a computer-readable tangible storage device or a computer-readable physical storage device. In other words, computer-readable storage media 1224 is media that can be touched by a person.

Alternatively, program code 1218 may be transferred to data processing system 1200, e.g., remotely over a network, using computer-readable signal media 1226. Computer-readable signal media 1226 may be, for example, a propagated data signal containing program code 1218. For example, computer-readable signal media 1226 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 1218 may be downloaded over a network to persistent storage 1208 from another device or data processing system through computer-readable signal media 1226 for use within data processing system 1200. For instance, program code stored in a computer-readable storage medium in a server data processing system may be downloaded over a network from the server to data processing system 1200. The computer providing program code 1218 may be a server computer, a client computer, or some other device capable of storing and transmitting program code 1218.

In some examples, program code 1218 may comprise an operating system (OS) 1250. Operating system 1250, which may be stored on persistent storage 1208, controls and allocates resources of data processing system 1200. One or more applications 1252 take advantage of the operating system's management of resources via program modules 1254, and program data 1256 stored on storage devices 1216. OS 1250 may include any suitable software system configured to manage and expose hardware resources of computer 1200 for sharing and use by applications 1252. In some examples, OS 1250 provides application programming interfaces (APIs) that facilitate connection of different type of hardware and/or provide applications 1252 access to hardware and OS services. In some examples, certain applications 1252 may provide further services for use by other applications 1252, e.g., as is the case with so-called “middleware.” Aspects of present disclosure may be implemented with respect to various operating systems or combinations of operating systems.

The different components illustrated for data processing system 1200 are not meant to provide architectural limitations to the manner in which different embodiments may be implemented. One or more embodiments of the present disclosure may be implemented in a data processing system that includes fewer components or includes components in addition to and/or in place of those illustrated for computer 1200. Other components shown in FIG. 12 can be varied from the examples depicted. Different embodiments may be implemented using any hardware device or system capable of running program code. As one example, data processing system 1200 may include organic components integrated with inorganic components and/or may be comprised entirely of organic components (excluding a human being). For example, a storage device may be comprised of an organic semiconductor.

In some examples, processor unit 1204 may take the form of a hardware unit having hardware circuits that are specifically manufactured or configured for a particular use, or to produce a particular outcome or progress. This type of hardware may perform operations without needing program code 1218 to be loaded into a memory from a storage device to be configured to perform the operations. For example, processor unit 1204 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 1218) may be implemented as hardware, e.g., by specifying an FPGA configuration using a hardware description language (HDL) and then using a resulting binary file to (re)configure the FPGA.

In another example, data processing system 1200 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 1204 may be implemented using a combination of processors found in computers and hardware units. Processor unit 1204 may have a number of hardware units and a number of processors that are configured to run program code 1218. With this depicted example, some of the processes may be implemented in the number of hardware units, while other processes may be implemented in the number of processors.

In another example, system bus 1202 may comprise one or more buses, such as a system bus or an input/output bus. Of course, the bus system may be implemented using any suitable type of architecture that provides for a transfer of data between different components or devices attached to the bus system. System bus 1202 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI)).

Additionally, communications unit 1210 may include a number of devices that transmit data, receive data, or both transmit and receive data. Communications unit 1210 may be, for example, a modem or a network adapter, two network adapters, or some combination thereof. Further, a memory may be, for example, memory 1206, or a cache, such as that found in an interface and memory controller hub that may be present in system bus 1202.

### E. Illustrative Distributed Data Processing System

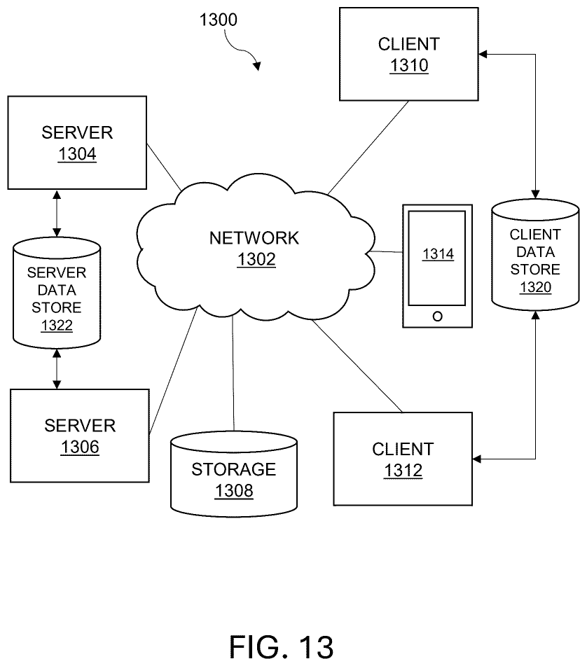

As shown in FIG. 13, this example describes a general network data processing system 1300, interchangeably termed a computer network, a network system, a distributed data processing system, or a distributed network, suitable for implementing embodiments of the Decision Support System described herein. For example, devices and applications of the present disclosure may communicate with each other and/or with the Internet via a network. For example, RAG-based LLM models may utilize API calls to access cloud-based models for inference purposes.

It should be appreciated that FIG. 13 is provided as an illustration of one implementation and is not intended to imply any limitation with regard to environments in which different embodiments may be implemented. Many modifications to the depicted environment may be made.

Network system 1300 is a network of devices (e.g., computers), each of which may be an example of data processing system 1200, and other components. Network data processing system 1300 may include network 1302, which is a medium configured to provide communications links between various devices and computers connected within network data processing system 1300. Network 1302 may include connections such as wired or wireless communication links, fiber optic cables, and/or any other suitable medium for transmitting and/or communicating data between network devices, or any combination thereof.

In the depicted example, a first network device 1304 and a second network device 1306 connect to network 1302, as do one or more computer-readable memories or storage devices 1308. Network devices 1304 and 1306 are each examples of data processing system 1200, described above. In the depicted example, devices 1304 and 1306 are shown as server computers, which are in communication with one or more server data store(s) 1322 that may be employed to store information local to server computers 1304 and 1306, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 1310 and 1312 and/or a client smart device 1314, may connect to network 1302. Each of these devices is an example of data processing system 1200, described above regarding FIG. 12. Client electronic devices 1310, 1312, and 1314 may include, for example, one or more personal computers, network computers, and/or mobile computing devices such as personal digital assistants (PDAs), smart phones, handheld gaming devices, wearable devices, and/or tablet computers, and the like. In the depicted example, server 1304 provides information, such as boot files, operating system images, and applications to one or more of client electronic devices 1310, 1312, and 1314. Client electronic devices 1310, 1312, and 1314 may be referred to as “clients” in the context of their relationship to a server such as server computer 1304. Client devices may be in communication with one or more client data store(s) 1320, which may be employed to store information local to the clients (e,g., cookie(s) and/or associated contextual information). Network data processing system 1300 may include more or fewer servers and/or clients (or no servers or clients), as well as other devices not shown.

In some examples, first client electric device 1310 may transfer an encoded file to server 1304. Server 1304 can store the file, decode the file, and/or transmit the file to second client electric device 1312. In some examples, first client electric device 1310 may transfer an uncompressed file to server 1304 and server 1304 may compress the file. In some examples, server 1304 may encode text, audio, and/or video information, and transmit the information via network 1302 to one or more clients.

Client smart device 1314 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may include features of other known electronic devices, such as a media player, personal digital assistant, digital camera, video camera, and/or global positioning system. Smart devices (e.g., smartphones) may be capable of connecting with other smart devices, computers, or electronic devices wirelessly, such as through near field communications (NFC), BLUETOOTH©, WiFi, or mobile broadband networks. Wireless connectively may be established among smart devices, smartphones, computers, and/or other devices to form a mobile network where information can be exchanged.

Data and program code located in system 1300 may be stored in or on a computer-readable storage medium, such as network-connected storage device 1308 and/or a persistent storage 1208 of one of the network computers, as described above, and may be downloaded to a data processing system or other device for use. For example, program code may be stored on a computer-readable storage medium on server computer 1304 and downloaded to client 1310 over network 1302, for use on client 1310. In some examples, client data store 1320 and server data store 1322 reside on one or more storage devices 1308 and/or 1208.

Network data processing system 1300 may be implemented as one or more of different types of networks. For example, system 1300 may include an intranet, a local area network (LAN), a wide area network (WAN), or a personal area network (PAN). In some examples, network data processing system 1300 includes the Internet, with network 1302 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 1302 may be referred to as a “cloud.” In those examples, each server 1304 may be referred to as a cloud computing node, and client electronic devices may be referred to as cloud consumers, or the like. FIG. 13 is intended as an example, and not as an architectural limitation for any illustrative embodiments.

### F. Illustrative Combinations and Additional Examples

This section describes additional aspects and features of Decision Support Systems for Prescription Verification and fulfillment processes, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A0. A prescription fulfillment method, implemented in a data processing system, the method comprising:
utilizing one or more processors of the data processing system to:
   receive, from a medical provider, a request to fill a medical prescription for a patient, wherein the request includes prescription data associated with the medical prescription, patient data associated with the patient, and provider data associated with the provider; and
   perform a prescription verification process using a decision support system including one or more software programs including a plurality of instructions stored in a memory of the data processing system and executable by the one or more processors to:
      verify accuracy of the patient data, prescription data, and provider data;
      verify the medical prescription satisfies one or more policies and regulations;
      determine whether the medical prescription is safe to dispense to the patient based on the prescription data and the patient data; and
      generate an output recommendation indicating one or more of the following: the medical prescription is ready to fill without modification, the medical prescription requires clarification prior to filling, or a proposed modification to the medical prescription.

A1. The method of paragraph A0, wherein the decision support system includes one or more AI algorithms configured to be executed by the one or more processors to perform one or more steps of the prescription verification process, wherein the one or more AI algorithms include one or more of a decision tree, a predictive model, a large language model (LLM), and a rules-based engine.

A2. The method of paragraph A0 or A1, wherein the decision support system includes a Retrieval-Augmented Generation (RAG) based LLM.

A2.1. The method of paragraph A2, wherein the RAG based LLM is configured to reference one or more authoritative data sources and historical dispensing data when performing one or more steps of the prescription verification process.

A2.2. The method of paragraph A2 or A2.1, wherein the RAG based LLM is configured to generate and output the output recommendation.

A3. The method of any one of paragraphs A0-A2.2, wherein the prescription data includes a SIG of the prescription.

A3.1. The method of paragraph A3, wherein verifying accuracy of the prescription data includes verifying the SIG includes a dosage, a dose frequency, a route of administration, a duration of treatment, and a total medication quantity.

A3.2. The method of paragraph A3.1, wherein verifying accuracy of the prescription data includes checking consistency between the total medication quantity, the dose quantity, the dose frequency, and the duration of treatment.

A4. The method of any one of paragraphs A0-A3.2, wherein determining whether the prescription is safe to dispense includes performing a patient-agnostic safety check.

A4.1. The method of paragraph A4, wherein performing the patient-agnostic safety check includes determining whether a/the dose quantity and a/the dose frequency of the prescription data is within a safe dosage range for the medical prescription.

A4.2. The method of paragraph A4 or A4.1, wherein performing the patient-agnostic safety check further includes referencing one or more authoritative databases or historical dispensing data to determine the safe dosage range.

A4.3. The method of paragraph A4.1 or A4.2, further comprising in response to determining the dose quantity or dose frequency exceeds the safe dosage range, generating the output recommendation indicating the medical prescription requires clarification.

A4.4. The method of any one of paragraphs A4.1-A4.3, further comprising in response to determining the dose quantity or dose frequency exceeds the safe dosage range, generating the output recommendation indicating the proposed modification, wherein the proposed modification includes a modification of one or both of the dose quantity and dose frequency.

A5. The method of any one of paragraphs A0-A4.4, wherein determining whether the prescription is safe to dispense includes performing a patient-centric safety check.

A5.1. The method of paragraph A5, wherein performing the patient-centric safety check includes performing a drug utilization review (DUR) by analyzing the patient data and prescription data to identify adverse drug interactions, drug-disease contraindications, a drug abuse history of the patient, or drug-patient precautions based on an age, gender, or allergies of the patient.

A5.2. The method of paragraph A5.1, further comprising in response to identifying one or more of the adverse drug interactions, drug-disease contraindications, drug abuse history of the patient, or drug-patient precautions, generating the output recommendation indicating the medical prescription requires clarification.

A6. The method of any one of paragraphs A0-A5.2, wherein verifying the medical prescription satisfies required policies and regulations includes:
searching one or more regulatory databases to identify one or more jurisdiction-specific medication regulations relevant to the medical prescription; and
verifying the prescription data and patient data satisfies the one or more jurisdiction-specific medication regulations.

A6.1. The method of paragraph A6, wherein the jurisdiction-specific medication regulations are specific to a state in which the prescription is being filled or dispensed to.

A7. The method of any one of paragraphs A0-A6.1, wherein verifying the medical prescription satisfies required policies and regulations includes verifying the prescription data and patient data satisfies one or more pharmacy-specific regulations specific to a pharmacy filling the medical prescription.

A8. The method of any one of paragraphs A0-A7, further comprising prior to performing the prescription verification process, communicating the medical prescription to an insurance company of the patient, wherein the insurance company of the patient is included in the patient data.

A8.1. The method of paragraph A8, further comprising receiving confirmation of coverage of the medical prescription from the insurance company.

A8.2. The method of paragraph A8 or A8.1, further comprising receiving an insurance-company drug utilization review (DUR) of the patient from the insurance company.

A9. The method of any one of paragraphs A0-A8.2, further comprising prior to performing the prescription verification process, communicating a prescription confirmation request to the patient.

A9.1. The method of paragraph A9, further comprising performing the prescription verification process in response to receiving confirmation from the patient.

A10. The method of any one of paragraphs A0-A9.1, wherein verifying the accuracy of the patient data includes verifying the patient data includes a valid patient name, date of birth, and contact information.

A11. The method of any one of paragraphs A0-A10, wherein verifying the accuracy of the provider data includes verifying the provider data includes a valid provider name, provider contact information, and required prescriptive authority authorizing the provider to prescribe the medical prescription.

A12. The method of any one of paragraphs A0-A11, wherein verifying the accuracy of the patient data, provider data, and prescription data includes verifying the patient data provided by the provider in the request matches system patient data, system provider data, and system prescription data stored in a database of the decision support system.

A13. The method of any one of paragraphs A0-A12, further comprising in response to verifying the accuracy of the patient data, prescription data, and provider data, verifying the medical prescription satisfies one or more policies and regulations, and determining the medical prescription is safe to dispense to the patient, generating the output recommendation indicating the medical prescription is ready to fill without modification.

A14. The method of any one of paragraphs A0-A13, further comprising generating the output recommendation indicating the medical prescription requires clarification in response to determining one or more of the following: the patient data, prescription data, or provider data is not accurate, the medical prescription does not satisfy one or more of the policies and regulations, or the medical prescription is not safe to dispense to the patient.

A15. The method of any one of paragraphs A0-A14, further comprising in response to determining the patient data, prescription data, or provider data is not accurate, determining the medical prescription does not satisfy one or more of the policies and regulations, or determining the medical prescription is not safe to dispense to the patient, generating the output recommendation indicating the proposed modification to the medical prescription, wherein the proposed modification includes a modification to one or more of the patient data, prescription data, or provider data.

A16. The method of any one of paragraphs A0-A15, wherein the data processing system further comprises a user interface, and wherein performing the prescription verification process further includes displaying the output recommendation on the user interface.

A17. The method of any one of paragraphs A0-A16, further comprising communicating with a server over a communication network to request data from one or more authoritative medical databases stored in a server-side database of the server when performing the prescription verification process.

A18. The method of any one of paragraphs A0-A17, wherein performing the prescription verification process further includes:
classifying the output recommendation as a high-confidence output recommendation or a low-confidence output recommendation; and
classifying the output recommendation as a high-risk output recommendation or a low-risk output recommendation.

A18.1. The method of paragraph B18, further comprising in response to classifying the output recommendation as a low-confidence output recommendation, performing the prescription verification process a second time.

A18.2. The method of paragraph A18 or A18.1, further comprising in response to classifying the output recommendation as a high-risk output recommendation, outputting the output recommendation to a pharmacist.

A18.3. The method of paragraph A18 or A18.1, further comprising in response to classifying the output recommendation as a low-risk output recommendation, generating a recommendation to bypass pharmacist inspection until after filling the medical prescription.

A19. The method of any one of paragraphs A0-A18, wherein performing the prescription verification further includes:
receiving an outcome result of the prescription verification process, wherein the outcome result indicates whether one or more actions were taken by a pharmacist reviewing the medical prescription;
determining whether the output recommendation generated by the decision processing system accurately predicted the one or more actions taken by the pharmacist;
classifying the output recommendation as a successful output recommendation or unsuccessful output recommendation based on whether the output recommendation accurately predicted the outcome result;
aggregating dispensing data including the output result, the output recommendation, and whether the recommendation was the successful output recommendation or the unsuccessful recommendation; and
retraining the one or more software programs of the decision processing system using the dispensing data.

B0. A prescription verification system, comprising:
a data processing device including:
   a memory;
   one or more processors; and
   a decision support system including one or more software programs including a plurality of instructions stored in the memory and executable by the one or more processors to:
      receive a request to perform a prescription verification process for a medical prescription, wherein the request includes prescription data associated with the medical prescription, patient data associated with a patient of the medical prescription, and provider data associated with a medical provider of the medical prescription; and
      perform the prescription verification process including:
         verifying accuracy of the patient data, prescription data, and provider data;
         verifying the medical prescription satisfies one or more policies and regulations;
         determining whether the medical prescription is safe to dispense to the patient based on the prescription data and the patient data; and
         generating an output recommendation indicating one or more of the following: the medical prescription is ready to fill without modification, the medical prescription requires clarification prior to filling, or a proposed modification to the medical prescription.

B1. The system of paragraph B0, wherein the decision support system includes one or more AI algorithms configured to be executed by the one or more processors to perform one or more steps of the prescription verification process, wherein the one or more AI algorithms include one or more of a decision tree, a predictive model, a large language model (LLM), and a rules-based engine.

B2. The system of paragraph B0 or B1, wherein the decision support system includes a Retrieval-Augmented Generation (RAG) based LLM.

B2.1. The system of paragraph B2, wherein the RAG based LLM is configured to reference one or more authoritative data sources and historical dispensing data when performing one or more steps of the prescription verification process.

B2.2. The system of paragraph B2 or B2.1, wherein the RAG based LLM is configured to generate and output the output recommendation.

B3. The system of any one of paragraphs B0-B2.2, wherein the prescription data includes a SIG of the prescription.

B3.1. The system of paragraph B3, wherein verifying accuracy of the prescription data includes verifying the SIG includes a dosage, a dose frequency, a route of administration, a duration of treatment, and a total medication quantity.

B3.2. The system of paragraph B3.1, wherein verifying accuracy of the prescription data includes checking consistency between the total medication quantity, the dose quantity, the dose frequency, and the duration of treatment.

B4. The system of any one of paragraphs B0-B3.2, wherein determining whether the prescription is safe to dispense includes performing a patient-agnostic safety check.

B4.1. The system of paragraph B4, wherein performing the patient-agnostic safety check includes determining whether a/the dose quantity and a/the dose frequency of the prescription data is within a safe dosage range for the medical prescription.

B4.2. The system of paragraph B4 or B4.1, wherein performing the patient-agnostic safety check further includes referencing one or more authoritative databases or historical dispensing data to determine the safe dosage range.

B4.3. The system of paragraph B4.1 or B4.2, further comprising in response to determining the dose quantity or dose frequency exceeds the safe dosage range, generating the output recommendation indicating the medical prescription requires clarification.

B4.4. The system of any one of paragraphs B4.1-B4.3, further comprising in response to determining the dose quantity or dose frequency exceeds the safe dosage range, generating the output recommendation indicating the proposed modification, wherein the proposed modification includes a modification of one or both of the dose quantity and dose frequency.

B5. The system of any one of paragraphs B0-B4.4, wherein determining whether the prescription is safe to dispense includes performing a patient-centric safety check.

B5.1. The system of paragraph B5, wherein performing the patient-centric safety check includes performing a drug utilization review (DUR) by analyzing the patient data and prescription data to identify adverse drug interactions, drug-disease contraindications, a drug abuse history of the patient, or drug-patient precautions based on an age, gender, or allergies of the patient.

B5.2. The system of paragraph B5.1, further comprising in response to identifying one or more of the adverse drug interactions, drug-disease contraindications, drug abuse history of the patient, or drug-patient precautions, generating the output recommendation indicating the medical prescription requires clarification.

B6. The system of any one of paragraphs A0-A5.2, wherein verifying the medical prescription satisfies required policies and regulations includes:
searching one or more regulatory databases to identify one or more jurisdiction-specific medication regulations relevant to the medical prescription; and
verifying the prescription data and patient data satisfies the one or more jurisdiction-specific medication regulations.

B6.1. The system of paragraph B6, wherein the jurisdiction-specific medication regulations are specific to a state in which the prescription is being filled or dispensed to.

B7. The system of any one of paragraphs B0-B6.1, wherein verifying the medical prescription satisfies required policies and regulations includes verifying the prescription data and patient data satisfies one or more pharmacy-specific regulations specific to a pharmacy filling the medical prescription.

B8. The system of any one of paragraphs B0-B7, further comprising prior to performing the prescription verification process, communicating the medical prescription to an insurance company of the patient, wherein the insurance company of the patient is included in the patient data.

B8.1. The system of paragraph B8, further comprising receiving confirmation of coverage of the medical prescription from the insurance company.

B8.2. The system of paragraph B8 or B8.1, further comprising receiving an insurance-company drug utilization review (DUR) of the patient from the insurance company.

B9. The system of any one of paragraphs B0-B8.2, further comprising prior to performing the prescription verification process, communicating a prescription confirmation request to the patient.

B9.1. The system of paragraph B9, further comprising performing the prescription verification process in response to receiving confirmation from the patient.

B10. The system of any one of paragraphs B0-B9.1, wherein verifying the accuracy of the patient data includes verifying the patient data includes a valid patient name, date of birth, and contact information.

B111. The system of any one of paragraphs B0-B10, wherein verifying the accuracy of the provider data includes verifying the provider data includes a valid provider name, provider contact information, and required prescriptive authority authorizing the medical provider to prescribe the medical prescription.

B12. The system of any one of paragraphs B0-B111, wherein verifying the accuracy of the patient data, provider data, and prescription data includes verifying the patient data provided by the medical provider in the request matches system patient data, system provider data, and system prescription data stored in a database of the decision support system.

B13. The system of any one of paragraphs B0-B12, further comprising in response to verifying the accuracy of the patient data, prescription data, and provider data, verifying the medical prescription satisfies one or more policies and regulations, and determining the medical prescription is safe to dispense to the patient, generating the output recommendation indicating the medical prescription is ready to fill without modification.

B14. The system of any one of paragraphs B0-B13, further comprising generating the output recommendation indicating the medical prescription requires clarification in response to determining one or more of the following: the patient data, prescription data, or provider data is not accurate, the medical prescription does not satisfy one or more of the policies and regulations, or the medical prescription is not safe to dispense to the patient.

B15. The system of any one of paragraphs B0-B14, further comprising in response to determining the patient data, prescription data, or provider data is not accurate, determining the medical prescription does not satisfy one or more of the policies and regulations, or determining the medical prescription is not safe to dispense to the patient, generating the output recommendation indicating the proposed modification to the medical prescription, wherein the proposed modification includes a modification to one or more of the patient data, prescription data, or provider data.

B16. The system of any one of paragraphs B0-B15, wherein the data processing system further comprises a user interface, and wherein performing the prescription verification process further includes displaying the output recommendation on the user interface.

B17. The system of any one of paragraphs B0-B16, wherein the data processing system further comprises one or more aggregated dispensing databases storing historical prescription dispensing data, and wherein the decision processing system is configured to reference data stored in the one or more aggregated dispensing databases when performing the prescription verification process.

B18. The system of any one of paragraphs B0-B17, wherein the data processing system is configured to communicate with a server over a communication network, wherein the decision processing system is configured to request data from one or more authoritative medical databases stored in a server-side database of the server when performing the prescription verification process.

B19. The system of any one of paragraphs B0-B18, wherein performing the prescription verification process by the one or more processors further includes:
classifying the output recommendation as a high-confidence output recommendation or a low-confidence output recommendation; and
classifying the output recommendation as a high-risk output recommendation or a low-risk output recommendation.

B19.1. The system of paragraph B19, further comprising in response to classifying the output recommendation as a low-confidence output recommendation, performing the prescription verification process a second time.

B19.2. The system of paragraph B19 or B19.1, further comprising in response to classifying the output recommendation as a high-risk output recommendation, outputting the output recommendation to a pharmacist.

B19.3. The system of paragraph B19 or B19.1, further comprising in response to classifying the output recommendation as a low-risk output recommendation, generating a recommendation to bypass pharmacist inspection until after filling the medical prescription.

B20. The system of any one of paragraphs B0-B18, wherein performing the prescription verification process by the one or more processors further includes:
receiving an outcome result of the prescription verification process, wherein the outcome result indicates whether one or more actions were taken by a pharmacist reviewing the medical prescription;
determining whether the output recommendation generated by the decision processing system accurately predicted the one or more actions taken by the pharmacist;
classifying the output recommendation as a successful output recommendation or unsuccessful output recommendation based on whether the output recommendation accurately predicted the outcome result;
aggregating dispensing data including the output result, the output recommendation, and whether the recommendation was the successful output recommendation or the unsuccessful recommendation; and
retraining the one or more software programs of the decision processing system using the dispensing data.

C0. A prescription verification system, comprising:
one or more data processing systems including:
   a memory;
   one or more processors; and
   a decision support system including one or more software programs including a plurality of instructions stored in the memory and executable by the one or more processors to:
      receive a request to perform a prescription verification precheck for a medical prescription, wherein the request includes prescription data associated with the medical prescription, patient data associated with a patient of the medical prescription, and provider data associated with a medical provider of the medical prescription; and
      perform the prescription verification precheck to determine whether the medical prescription is in a satisfactory condition to be filled or whether the medical prescription is in an unsatisfactory condition and requires review by a user prior to being filled, wherein performing the prescription verification precheck includes:
         verifying accuracy of the patient data, the prescription data, and the provider data;
         verifying the medical prescription satisfies one or more policies and regulations; and
         performing a prescription safety check to determine whether the medical prescription is safe to dispense to the patient based on the prescription data and the patient data.

C1. The system of paragraph C0, wherein the decision support system includes one or more artificial intelligence (AI) algorithms configured to be executed by the one or more processors to perform one or more steps of the prescription verification precheck, wherein the one or more AI algorithms include one or more of a decision tree, a predictive model, a large language model (LLM), and a rules-based engine.

C2. The system of paragraph C0 or C1, wherein performing the prescription verification precheck by the decision support system further includes:
identifying one or more inaccuracies in the patient data, the prescription data, or the provider data; and
determining a prescription edit to resolve the one or more inaccuracies, such that the medical prescription is in the satisfactory condition.

C2.1. The system of paragraph C2, wherein performing the prescription verification precheck further comprises:
determining a confidence level of the prescription edit; and
in response to determining a high confidence level for the prescription edit, editing the medical prescription based on the prescription edit, such that the medical prescription is in the satisfactory condition; and
in response to determining a low confidence level for the prescription edit, determining the medical prescription requires the review by the user.

C3. The system of any one of paragraphs C0-C2.1, wherein the decision support system is further configured to:
generate an output assessment of the medical prescription based on the prescription verification precheck, wherein the output assessment indicates whether the medical prescription is in the satisfactory condition or the unsatisfactory condition; and
display the output assessment on a user interface of the one or more data processing systems.

C3.1. The system of paragraph C3, wherein the output assessment further includes one or more of a recommended user action, a proposed prescription edit, and a request for clarification of the medical prescription.

C4. The system of any one of paragraphs C0-C3.1, wherein the one or more processors are further configured to;
fill the medical prescription in response to the decision support system determining the medical prescription is in the satisfactory condition, wherein filling the medical prescription includes printing a prescription label for the medical prescription including the prescription data.

C5. The system of any one of paragraphs C0-C4, wherein performing the prescription safety check includes performing a patient-agnostic safety check including:
determining whether a dosage of the medical prescription is within a safe dosage range; and
in response to determining the dosage is not within the safe dosage range, determining the medical prescription fails the patient-agnostic safety check.

C6. The system of any one of paragraphs C0-C5, wherein performing the prescription safety check includes performing a patient-centric safety check including:
performing a drug utilization review (DUR) of the patient by analyzing the patient data and prescription data to identify adverse drug interactions, drug-disease contraindications, a drug abuse history of the patient, or drug-patient precautions based on an age, gender, or allergies of the patient; and
in response to identifying one or more of the adverse drug interactions, drug-disease contraindications, drug abuse history of the patient, or drug-patient precautions, determining the medical prescription fails the patient-centric safety check.

C7. The system of any one of paragraphs C0-C6, further comprising in response to determining the medical prescription fails the prescription safety check, determining the medical prescription requires a clarification from the medical provider or the patient prior to being filled.

C8. The system of any one of paragraphs C0-C7, further comprising the features of the system of any one of paragraphs B0-B20.

D0. A computer-implemented prescription fulfillment method, the method comprising:
utilizing one or more processors of a data processing system to:
   receive, from a medical provider, a request to fill a medical prescription for a patient, wherein the request includes prescription data associated with the medical prescription, patient data associated with the patient, and provider data associated with the medical provider; and
   perform a prescription verification precheck using a decision support system including one or more software programs including a plurality of instructions stored in a memory of the data processing system and executable by the one or more processors to:
      verify accuracy of the patient data, the prescription data, and the provider data;
      verify the medical prescription satisfies one or more policies and regulations;
      perform a prescription safety check to determine whether the medical prescription is safe to dispense to the patient based on the prescription data and the patient data; and
      determine whether the medical prescription is in a satisfactory condition to be filled or whether the medical prescription is in an unsatisfactory condition and requires review by a user prior to being filled.

D1. The method of paragraph D0, wherein the decision support system includes one or more artificial intelligence (AI) algorithms configured to be executed by the one or more processors to perform one or more steps of the prescription verification precheck, wherein the one or more AI algorithms include one or more of a decision tree, a predictive model, a large language model (LLM), and a rules-based engine.

D2. The method of paragraph D0 or D1, wherein performing the prescription verification precheck further includes:
identifying one or more inaccuracies in the patient data, the prescription data, or the provider data; and
determining a prescription edit to resolve the one or more inaccuracies, such that the medical prescription is in the satisfactory condition.

D2.1. The method of paragraph D2, wherein performing the prescription verification precheck further comprises:
determining a confidence level of the prescription edit; and
in response to determining a high confidence level for the prescription edit, editing the medical prescription based on the prescription edit, such that the medical prescription is in the satisfactory condition; and
in response to determining a low confidence level for the prescription edit, determining the medical prescription requires the review by the user prior to being filled.

D3. The method of any one of paragraphs D0-D2.1, further comprising:
generating an output assessment of the medical prescription based on the prescription verification precheck, wherein the output assessment indicates whether the medical prescription is in the satisfactory condition or the unsatisfactory condition; and
displaying the output assessment on a user interface of the data processing system.

D3.1. The method of paragraph D3, wherein the output assessment further includes one or more of a recommended user action, a proposed prescription edit, and a request for clarification of the medical prescription.

D4. The method of any one of paragraphs D0-D3.1, further comprising
in response to determining the medical prescription is in the satisfactory condition, filling the medical prescription; and
in response to determining the medical prescription is in the unsatisfactory condition, queueing the medical prescription for review by the user prior to being filled.

D4.1. The method of paragraph D4, wherein filling the medical prescription includes printing a prescription label including the prescription data of the medical prescription.

D5. The method of any one of paragraphs D0-D4.1, wherein performing the prescription safety check includes performing a patient-agnostic safety check and a patient-centric safety check.

D6. The method of any one of paragraphs D0-D5, further comprising in response to determining the medication prescription fails the prescription safety check, determining the medical prescription requires a clarification from the medical provider or the patient prior to being filled.

D7. The method of any one of paragraphs D0-D6, further comprising the method steps of any one of paragraphs A0-A19.

### Advantages, Features, and Benefits

The different embodiments and examples of the Decision Support System for Prescription Verification and fulfillment described herein provide several advantages over known solutions for prescription verification processes. For example, illustrative embodiments and examples described herein allow for the use of a plurality algorithms and/or AI tools to automatically perform one or more checks and/or verifications of the prescription in order to assist a pharmacist by eliminating noise & variability in the decision-making process as well as reducing the pharmacist's cognitive workload.

Additionally, and among other benefits, illustrative embodiments and examples described herein allow a Decision Support System that includes sophisticated algorithms that cross-reference multiple data points and leverage LLM-based review to ensure accuracy and efficiency in the evaluation process.

Additionally, and among other benefits, illustrative embodiments and examples described herein allow a Decision Support System to perform a Prescription Verification Precheck to determine whether the prescription is in satisfactory condition to be filled or whether the prescription can be edited to be in satisfactory condition to be filled with a high degree of confidence. If the Decision Support System determines that the prescription is in the satisfactory condition, a Pharmacist Verification 1 (PV1) process may be postponed until after the prescription is filled. This streamlines the prescription fulfillment workflow by not requiring review of the prescription by a pharmacist both before and after the prescription is physically filled.

Additionally, and among other benefits, illustrative embodiments and examples described herein allow a Decision Support System that seamlessly integrates with existing pharmacy management systems to provide a streamlined workflow, minimizing the need for manual intervention.

Additionally, and among other benefits, illustrative embodiments and examples described herein allow a Decision Support System that utilizes natural language processing, such as an LLM, to craft recommendations to the pharmacist that assist the pharmacist in the Prescription Verification process.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A prescription verification system, comprising:
one or more data processing systems including:
a memory;
one or more processors; and
a decision support system including one or more software programs including a plurality of instructions stored in the memory and executable by the one or more processors to:
receive a request to perform a prescription verification precheck for a medical prescription, wherein the request includes prescription data associated with the medical prescription, patient data associated with a patient of the medical prescription, and provider data associated with a medical provider of the medical prescription; and
perform the prescription verification precheck to determine whether the medical prescription is in a satisfactory condition to be filled or whether the medical prescription is in an unsatisfactory condition and requires review by a user prior to being filled, wherein performing the prescription verification precheck includes:
verifying accuracy of the patient data, the prescription data, and the provider data;
verifying the medical prescription satisfies one or more policies and regulations; and
performing a prescription safety check to determine whether the medical prescription is safe to dispense to the patient based on the prescription data and the patient data.

2. The system of claim 1, wherein the decision support system includes one or more artificial intelligence (AI) algorithms configured to be executed by the one or more processors to perform one or more steps of the prescription verification precheck, wherein the one or more AI algorithms include one or more of a decision tree, a predictive model, a large language model (LLM), and a rules-based engine.

3. The system of claim 1, wherein performing the prescription verification precheck by the decision support system further includes:
identifying one or more inaccuracies in the patient data, the prescription data, or the provider data; and
determining a prescription edit to resolve the one or more inaccuracies, such that the medical prescription is in the satisfactory condition.

4. The system of claim 3, wherein performing the prescription verification precheck further comprises:
determining a confidence level of the prescription edit; and
in response to determining a high confidence level for the prescription edit, editing the medical prescription based on the prescription edit, such that the medical prescription is in the satisfactory condition; and
in response to determining a low confidence level for the prescription edit, determining the medical prescription requires the review by the user.

5. The system of claim 1, wherein the decision support system is further configured to:
generate an output assessment of the medical prescription based on the prescription verification precheck, wherein the output assessment indicates whether the medical prescription is in the satisfactory condition or the unsatisfactory condition; and
display the output assessment on a user interface of the one or more data processing systems.

6. The system of claim 5, wherein the output assessment further includes one or more of a recommended user action, a proposed prescription edit, and a request for clarification of the medical prescription.

7. The system of claim 1, wherein the one or more processors are further configured to fill the medical prescription in response to the decision support system determining the medical prescription is in the satisfactory condition, wherein filling the medical prescription includes printing a prescription label for the medical prescription including the prescription data.

8. The system of claim 1, wherein performing the prescription safety check includes performing a patient-agnostic safety check including:
determining whether a dosage of the medical prescription is within a safe dosage range; and
in response to determining the dosage is not within the safe dosage range, determining the medical prescription fails the patient-agnostic safety check.

9. The system of claim 1, wherein performing the prescription safety check includes performing a patient-centric safety check including:
performing a drug utilization review (DUR) of the patient by analyzing the patient data and prescription data to identify adverse drug interactions, drug-disease contraindications, a drug abuse history of the patient, or drug-patient precautions based on an age, gender, or allergies of the patient; and
in response to identifying one or more of the adverse drug interactions, drug-disease contraindications, drug abuse history of the patient, or drug-patient precautions, determining the medical prescription fails the patient-centric safety check.

10. The system of claim 1, further comprising in response to determining the medical prescription fails the prescription safety check, determining the medical prescription requires a clarification from the medical provider or the patient prior to being filled.

11. A computer-implemented prescription fulfillment method, the method comprising:
utilizing one or more processors of a data processing system to:
receive, from a medical provider, a request to fill a medical prescription for a patient, wherein the request includes prescription data associated with the medical prescription, patient data associated with the patient, and provider data associated with the medical provider; and
perform a prescription verification precheck using a decision support system including one or more software programs including a plurality of instructions stored in a memory of the data processing system and executable by the one or more processors to:
verify accuracy of the patient data, the prescription data, and the provider data;
verify the medical prescription satisfies one or more policies and regulations;
perform a prescription safety check to determine whether the medical prescription is safe to dispense to the patient based on the prescription data and the patient data; and
determine whether the medical prescription is in a satisfactory condition to be filled or whether the medical prescription is in an unsatisfactory condition and requires review by a user prior to being filled.

12. The method of claim 11, wherein the decision support system includes one or more artificial intelligence (AI) algorithms configured to be executed by the one or more processors to perform one or more steps of the prescription verification precheck, wherein the one or more AI algorithms include one or more of a decision tree, a predictive model, a large language model (LLM), and a rules-based engine.

13. The method of claim 11, wherein performing the prescription verification precheck further includes:
identifying one or more inaccuracies in the patient data, the prescription data, or the provider data; and
determining a prescription edit to resolve the one or more inaccuracies, such that the medical prescription is in the satisfactory condition.

14. The method of claim 13, wherein performing the prescription verification precheck further comprises:
determining a confidence level of the prescription edit; and
in response to determining a high confidence level for the prescription edit, editing the medical prescription based on the prescription edit, such that the medical prescription is in the satisfactory condition; and
in response to determining a low confidence level for the prescription edit, determining the medical prescription requires the review by the user prior to being filled.

15. The method of claim 11, further comprising:
generating an output assessment of the medical prescription based on the prescription verification precheck, wherein the output assessment indicates whether the medical prescription is in the satisfactory condition or the unsatisfactory condition; and
displaying the output assessment on a user interface of the data processing system.

16. The method of claim 15, wherein the output assessment further includes one or more of a recommended user action, a proposed prescription edit, and a request for clarification of the medical prescription.

17. The method of claim 11, further comprising
in response to determining the medical prescription is in the satisfactory condition, filling the medical prescription; and
in response to determining the medical prescription is in the unsatisfactory condition, queueing the medical prescription for review by the user prior to being filled.

18. The method of claim 17, wherein filling the medical prescription includes printing a prescription label including the prescription data of the medical prescription.

19. The method of claim 11, wherein performing the prescription safety check includes performing a patient-agnostic safety check and a patient-centric safety check.

20. The method of claim 11, further comprising in response to determining the medication prescription fails the prescription safety check, determining the medical prescription requires a clarification from the medical provider or the patient prior to being filled.
